SEQUENCE LISTING (15 sequences)

SEQ ID NO: 1 - protein, 166 aa, Artificial Sequence
  Charge-modified myoglobin
MHHHHHHGSSGENLYFQGLSDGEWQLVLNVWGKVEADIPGHGQEVLIRLFKGHPKTLKKFDRFKHLKSKKKMKASEKLKKHGATVLTALGGILKKKGHHEAKIKPLAQSHATKHKIPVKYLKFISKAIIKVLQSKHPGDFGKKAQGAMNKALKLFRKKMASNYKEL

SEQ ID NO: 2 - protein, 166 aa, Artificial Sequence
  Charge-modified myoglobin
MHHHHHHGSSGENLYFQGLSDGEWQLVLNVWGKVEAKIPGHGQEVLIRLFKGHPKTLKKFDRFKHLKSKKKMKASKKLKKHGATVLTALGGILKKKGHHKAKIKPLAQSHATKHKIPVKYLKFISKAIIQVLQSKHPGKFGAKAQGAMNKALKLFRKKMASNYKKL

SEQ ID NO: 3 - protein, 166 aa, Artificial Sequence
  Charge-modified myoglobin
MHHHHHHGSSGENLYFQGLSKGKWQLVLNVWGKVKAKIPGHGQKVLIRLFKGHPKTLKEFKRFKHLKSKKKMKASKKLKKHGATVLTALGGILKKKGHHEAKIEPLAQSHATEHEIPVEYLEFISKAIIQVLQSKHPGKFGAKAQGAMNKALKLFRKDMASNYEEL

SEQ ID NO: 4 - protein, 171 aa, Artificial Sequence
  Charge-modified myoglobin
MHHHHHHGGGGSENLYFQGLSDGEWQLVLNVWGKVEADIPGHGQEVLIRLFKGHPETLKKFDRFKKLKSEDKMKKSEDLKKHGATVLKRLGGILKKKGRHEAKIKPLAQRHAKKHKIPVKYLEFRSEAIIRVLRSKHPGDFGADAQGAMNKALELFRKDMASNYKELGFQG

SEQ ID NO: 5 - protein, 171 aa, Artificial Sequence
  Charge-modified myoglobin
MHHHHHHGGGGSENLYFQGLSKGEWKLVLNVWGKVEADIPGHGQEVLIRLFKGHPKTLKKFKRFKHLKSEKKMKASKDLKKHGATVLTALGGILKKKGHHKAEIKPLAQSHATKHKIPVKYLEFISEAIIQVLQSKHPGKFGAKAQGAMNKALELFRKDMASNYKKLGFQG

SEQ ID NO: 6 - protein, 171 aa, Artificial Sequence
  Charge-modified myoglobin
MHHHHHHGGGGSENLYFQGLSDGEWRLVLKVWGKVERDIPGHGQEVLIRLFKGHPETLKKFDRFKHLKSEREMKASKDLKKHGATVLTALGGILKKKGHHEAEIKPLAKSHATKHKIPVKYLKFISKAIIQVLQSKHPGDFGARAQGAMNKALELFRKDMARNYKKLGFQG

SEQ ID NO: 7 - protein, 169 aa, Artificial Sequence
  Charge-modified myoglobin
MHHHHHHGGGGSENLYGLSDGEWRLVLKVWGKVERDIPGHGQEVLIRLFKGHPETLKKFDRFKHLKSRDEMKASEKLKKHGATVLTALGGILKKKGHHEAEIKPLAKSHATKHKIPVKYLKFISKAIIQVLQSKHPGDFGADAQGAMNKALELFRKDMASKYKKLGFQG

SEQ ID NO: 8 - protein, 163 aa, Artificial Sequence
  Charge-modified myoglobin
MHHHHHHGSGGLSDGEWQLVLKVWGKVEADIPGHGQEVLIRLFKGHPETLEKFDRFKHLKSEDEMKASEDLKKHGATVLTALGKILKKKGHHEAEIKPLAQSHATKHKIPVKYLKFISEAIIKVLQSKHPGDFGADAQGAMKKALELFRKDMASKYKELGFQG

SEQ ID NO: 9 - protein, 163 aa, Artificial Sequence
  Charge-modified myoglobin
MHHHHHHGSGGLSDGEWQLVLKVWGKVEADIPGHGQEVLIRLFKGHPETLKKFDRFKHLKSEDEMKASEDLKKHGATVLKKLGKILKKKGKHEAEIKPLAQSHATKHKIPVKYLKFISEAIIKVLQSKHPGDFGADAQGAMKKALKLFRKDMASKYKELGFQG

SEQ ID NO: 10 - protein, 163 aa, Artificial Sequence
  Charge-modified myoglobin
MHHHHHHGSGGLSDGEWQLVLKVWGKVEADIPGHGQEVLIRLFKGHPETLEKFDRFKHLKSEDEMKRSEDLKKHGATVLKALGKILKKKGHHEAEIKPLAQSHATKHKIPVKYLKFISEAIIKVLQSKHPGDFGADAQGAMKKALELFRKDMASKYKELGFQG

SEQ ID NO: 11 - protein, 163 aa, Artificial Sequence
  Charge-modified myoglobin
MHHHHHHGSGGLSDGEWQLVLKVWGKVEADIPGHGQEVLIRLFKGHPETLEKFDRFKKLKSEDEMKRSEDLKKHGATVLKKLGKILKKKGKHEAEIKPLAQSHATKHKIPVKYLKFISEAIIKVLQSKHPGDFGADAQGAMKKALKLFRKDMASKYKELGFQG

SEQ ID NO: 12 - protein, 163 aa, Artificial Sequence
  Charge-modified myoglobin
MHHHHHHGSGGLRDGEWQLVLKVWGKVEADIPGHGQEVLIRLFKGHPETLEKFDRFKKLKSEDEMKRSEDLKKHGATVLKKLGKILKKKGKHEAEIKPLAQSHATKHKIPVKYLKFISEAIIKVLQSKHPGDFGADAQGAMKKALKLFRKDMASKYKELGFQG

SEQ ID NO: 13 - protein, 695 aa, Artificial Sequence
  Charge-modified myoglobin
MHHHHHHKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGENLYFQGSGWNPPTFSPALLVVTEGDNATFTCSFSNTSESFVLNWYRMSPSNQTVKLAAFPEDRSQPGQDSRFRVTQLPNGRDFHMSVVRARRNDSGTYLCSAISLAPKAQIKESLRAELRVTERRAEVPTAHPSPSPGGGGSGGGGSGGGGSGLSDGEWQLVLKVWGKVEADIPGHGQEVLIRLFKGHPETLKKFDRFKHLKSEDEMKASEDLKKHGATVLKKLGKILKKKGKHEAEIKPLAQSHATKHKIPVKYLKFISEAIIKVLQSKHPGDFGADAQGAMKKALKLFRKDMASKYKELGFQG

SEQ ID NO: 14 - protein, 689 aa, Artificial Sequence
  Charge-modified myoglobin
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGENLYFQGSGWNPPTFSPALLVVTEGDNATFTCSFSNTSESFVLNWYRMSPSNQTVKLAAFPEDRSQPGQDSRFRVTQLPNGRDFHMSVVRARRNDSGTYLCSAISLAPKAQIKESLRAELRVTERRAEVPTAHPSPSPGGGGSGGGGSGGGGSGLSDGEWQLVLKVWGKVEADIPGHGQEVLIRLFKGHPETLKKFDRFKHLKSEDEMKASEDLKKHGATVLKKLGKILKKKGKHEAEIKPLAQSHATKHKIPVKYLKFISEAIIKVLQSKHPGDFGADAQGAMKKALKLFRKDMASKYKELGFQG

SEQ ID NO: 15 - protein, 239 aa, Artificial Sequence
  Charge-modified GFP
MASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

(19) United States
(12) Patent Application Publication
(10) Pub. No.: US 2022/0127317 A1
(43) Pub. Date: Apr. 28, 2022

(54) ANTITUMOR CELL COMPRISING A CHARGE MODIFIED GLOBIN

(71) Applicant: CYTOSEEK LTD, Bristol (GB)

(72) Inventors: Adam Willis PERRIMAN, Bristol (GB); Benjamin Michael CARTER, Bristol (GB); Thomas Iain Phillip GREEN, Bristol (GB); David COE, Bristol (GB); William Hongyu ZHANG, Bristol (GB)

(21) Appl. No.: 17/436,244

(22) PCT Filed: Mar. 6, 2019
(86) PCT No.: PCT/GB2020/050537

§ 371 Date: Sep. 3, 2021

(30) Foreign Application Priority Data
Mar. 6, 2019 (GB) 1902992.5

(51) Int. Cl. C07K 14/47; A61K 9/127; A61P 35/00; C12N 5/0783

(52) U.S. Cl. CPC C07K 14/4716; A61K 9/127; A61K 38/00; C12N 5/0636; A61P 35/00

(57) ABSTRACT

There is provided an antitumour cell, liposome or micelle, comprising at least one charge-modified globin associated with the membrane of the cell, liposome or micelle, and methods of making and using the same.

### TECHNICAL FIELD

This invention relates to antitumour cells, liposomes and micelles comprising a charge-modified globin associated with the membrane of the cell, liposome or micelle in order to enhance the antitumour properties of the cell, liposome or micelle. Methods of production and use are also provided.

### BACKGROUND

A number of antitumour therapeutics involve the use of cells, liposomes and micelles. Recent examples include treatment with engineered immune cells such as CAR-T cells, which are cytotoxic T cells that have been genetically engineered to express a chimeric antigen receptor (CAR) with tumour specificity. The CAR allows the T cell to bind to a tumour cell, where the T cell can then kill the tumour cell. Treatment can also be carried out using liposomes and micelles that, in a manner having similarities with cytotoxic T cells, bind to tumours and deliver antitumour compositions.

These efforts are, however, frustrated by the ability of a tumour to evade the immune system. The solid tumour microenvironment is highly immunosuppressive: tumour cells express checkpoint inhibitors and recruit suppressive cell populations, and localised hypoxia causes a cascade of immunosuppressive gene expression. One effect of this is that cancer killing cells such as cytotoxic T cells are ‘switched off’, i.e. they lose their ability to identify and kill tumour cells.

Current approaches towards overcoming this immune suppression include systemic administration of antibodies that block proteins (such as PD-1, PD-L1 and CTLA4) that deactivate cytotoxic T-cells, depleting immunosuppressive regulatory T-cells (a type of immunosuppressive T-cell), and use of oxygen-enriched air or erythropoietin to increase oxygen levels. It has also been reported that hypoxia in cancer cells can be reduced by administration of globins, which can be administered to try and enhance the effectiveness of a chemotherapeutic drug (for example, as described in US2015/0374796).

The present invention provides antitumour cells, liposomes and micelles where the antitumour activity of the cells, liposomes and micelles is enhanced, in particular by reducing the ability of a tumour to evade the antitumour effects of the antitumour cells, liposomes and micelles as well as reducing the ability of a tumour to evade the immune response.

### SUMMARY OF THE INVENTION

According to a first aspect, the invention provides an antitumour cell, antitumour liposome or antitumour micelle, comprising at least one charge-modified globin associated with the membrane of the cell, liposome or micelle.

The inventors have identified an improved therapeutic composition for the treatment of tumours, in particular solid tumours. Surprisingly, charge-modified globins were found to successfully associate with the membranes of cells, liposomes and micelles, as well as to successfully retain their oxygen transport and delivery function despite being charge-modified and associated with a cell, liposome or micelle. Furthermore, this association was found to occur without losing key characteristics of the cell, liposome or micelle, such as stability, viability and activity. Specifically, experiments on T cells showed that viability, proliferation ability and, critically, activity, were not lost. An important aspect of associating a charge-modified globin with a membrane of an antitumour cell, liposome or micelle is that the charge-modified globin can simultaneously provide beneficial effects to the cell, liposome or micelle and to the tumour cells. For example, as discussed in detail below, the charge-modified globin can tune and/or improve activity of antitumour cells against hypoxic solid tumour cells. Furthermore, the charge-modified globin can also reduce hypoxia in a hypoxic solid tumour, thus alleviating the effects of the hypoxic conditions. For example, reduction of hypoxia can sensitise the tumour cells to the effects of antitumour cells and/or chemotherapeutic treatments that may be delivered by liposomes or micelles. The cells, liposomes or micelles of the invention have therefore been equipped with the inherent ability to overcome the tumour's ability to evade the antitumour effects of the cells, liposomes and micelles, as well as reducing the tumour's ability to evade any immune response concurrent with the effects of the antitumour cells, liposomes or micelles of the invention. Association of the charge-modified globin with the membrane of the cell, liposome or micelle, means that the local concentration of the globin in the region of the cell, liposome or micelle could only be matched in a non-associated system by systemic administration of large excesses of globin. Where the cell, liposome or micelle targets hypoxic solid tumours, this also applies to the local concentration of the globin in the region of hypoxic solid tumour. Furthermore, by associating the charge-modified globin with the antitumour cell, liposome or micelle, the antitumour effects of the cell, liposome or micelle against a solid tumour are inherently occurring at the same time as the globin is acting against the solid tumour. Yet further, where the antitumour cell, liposome or micelle is associated with and acting against a specific section within a solid tumour, the associated charge-modified globin is inherently also acting against that same section of the solid tumour.

The globin is charge-modified. This means that the net surface charge of the globin is modified with respect to the native (or “unmodified” or “wild-type”) globin. In other words, at least one residue that was negatively charged or neutral in the native protein has been modified to bear a positive charge, or at least one residue that was positively charged or neutral in the native protein has been modified to bear a negative charge. A “residue bearing a charge” can be understood to include a residue that is inherently charged (for example, the proteinogenic amino acids glutamate, aspartate, arginine, lysine and histidine), and a residue that is charged because it has undergone a modification to insert at least one functional group bearing one or more charges.

Typically, the charge is assessed at physiological pH, for example at about pH 6-9, for example about pH 6, 6.5, 7, 7.5, 8, 8.5 or about 9.

The charge modification of the globin can be brought about by various means. For instance, the native globin protein can be provided, and then the protein chemically modified to change the charge state of one or more residues. Alternatively, a charge-modified globin can be generated by recombinant expression of a sequence that encodes a charge-modified globin. A charge-modified globin can also be generated by a combination of expressing a charge-modified globin, and chemical modification.

As described above, the charge-modified globin has one or more charge modifications with respect to the native protein. For example, the number of residues that have been modified to bear one or more positive charges can be 1 to 100, for example, 1 to 80, 10 to 70, 20 to 60, or 30 to 50, such as about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55. In addition, or alternatively, the number of residues that have been modified to bear one or more negative charges can be 1 to 100, for example, 1 to 80, 10 to 70, 20 to 60, or 30 to 50, such as about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55.

The charge-modified globin may be referred to as a cationised globin in the case of modification to have an overall increased surface positive charge, or as an anionised globin in the case of modification to have an overall increased surface negative charge. For a cationised globin the overall change in surface positive charge may be +1 to +100, for example, +1 to +80, +10 to +70, +20 to +60, or +30 to +50, such as about +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, or +55. The overall surface positive charge of the cationised globin may be +1 to +100, for example, +1 to +80, +10 to +70, +20 to +60, or +30 to +50, such as at least about +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, or +55.

For an anionised globin the overall change in surface negative charge may be −1 to −100, for example, −1 to −80, −10 to −70, −20 to −60, or −30 to −50, such as about −5, −6, −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54 or −55. The overall surface negative charge of the anionised globin may be −1 to −100, for example, −1 to −80, −10 to −70, −20 to −60, or −30 to −50, such as at least about −5, −6, −7, −8, −9, −10, −11, −12, −13, −14, −15, −16, −17, −18, −19, −20, −21, −22, −23, −24, −25, −26, −27, −28, −29, −30, −31, −32, −33, −34, −35, −36, −37, −38, −39, −40, −41, −42, −43, −44, −45, −46, −47, −48, −49, −50, −51, −52, −53, −54 or −55.

Typically, the charge-modified globin comprises a percentage of residues bearing a positive or negative charge, determined as a percentage of the total number of amino acid residues in the protein. The percentage for either positive or negative charges is greater than the percentage of either positive or negative charges in the corresponding native globin. For example, the native globin may have 5.0-40% of its total amino acid residues as positively charged residues and the charge-modified globin may have a higher percentage than in the corresponding native globin. For example, native human myoglobin has 14% of its total amino acid residues as positively charged residues. Likewise, human haemoglobin has 10% of its total amino acid residues as positively charged residues, whilst horse heart myoglobin and chimpanzee myoglobin both have 14%. The charge-modified globin may have at least about 18%, 19%, 20%, 21%, 22%, 23%, 24%, 25%, 26%, 27%, 28%, 29% or at least about 30% of its total amino acid residues as positively charged residues. The same applies for negatively charged residues.

The charge-modified globin can also be referred to as a supercharged globin. The term “supercharged globin” used herein may, therefore, refer to any globin having one or more charge modifications with respect to the native protein, as already described. The generation of supercharged proteins is known in the art. Examples of the production of such a supercharged protein, in the context of Green Fluorescent Protein (GFP), are disclosed in Lawrence et al. (J. Am. Chem. Soc. (2007) vol. 129 p.10110-10112). Such so-called “supercharged” proteins have previously been used to facilitate delivery of molecules through the phospholipid bilayer cell membrane to the interior of a cell (Zang et al. (2017) PLoS One 12(6):e0180138; WO2009/134808; WO2010/129023; WO2016/069910; Thompson et al. (2012) Methods Enzymol. vol. 503 p. 293-319; McNaughton et al. (2009) Proc. Natl. Acad. Sci. U.S.A. vol. 106 p. 6111-6116). It is, therefore, wholly surprising that, when used for charge-modified globins and charge-modified globins comprising a polymer surfactant coating as described herein, an antitumour cell, liposome or micelle, comprising at least one charge-modified globin associated with the membrane of the cell, liposome or micelle can be obtained.

The charge-modified globin is associated with the membrane of the cell, liposome or micelle. In one embodiment, the charge-modified globin is associated with the membrane by being bound to the membrane. This binding can be mediated by one or more covalent bonds and/or by one or more intermolecular forces, such as electrostatic forces, hydrogen bonding and/or hydrophobic interactions. The charge-modified globin can also, or alternatively, be associated with the membrane through being sterically locked in place. An advantage of associating with the membrane is that the globin can readily deliver oxygen to both the target tumour cell and to the antitumour cell, liposome or micelle. A further advantage is that the globin associated with the membrane can additionally serve as an anchor for a secondary antitumour molecule.

By “membrane”, we are referring to the structure that separates the interior of the cells, liposomes or micelles from the outside environment. In cells produced by natural organisms, the membrane is a phospholipid bilayer and is also known as the cell membrane, plasma membrane or cytoplasmic membrane. The charge-modified globin can be associated with the membrane by being embedded in the phospholipid bilayer (i.e. in the hydrophobic lipid region of the membrane) or can be associated with the exterior solvent-exposed surface of the phospholipid bilayer. This association with the solvent-exposed surface of the phospholipid bilayer can be mediated by binding directly with the hydrophilic phospholipid heads and/or by binding with cell-surface proteins. In a liposome, the membrane is the structure that is analogous to the cell membrane. In other words, the membrane is the outer membrane of the liposome. The liposome membrane can comprise a phospholipid bilayer. It is noted, however, that liposome membranes can comprise bilayers formed of different amphiphilic molecules, as is known in the art. In a micelle, the membrane is the outer perimeter of the micelle, which is formed of the hydrophobic heads of an amphiphilic molecule, such as a phospholipid.

In a preferred embodiment, the charge-modified globin is embedded in the membrane or is associated with (e.g. bound to) the exterior (i.e., solvent-exposed) surface of the membrane.

The membrane of the cell preferably comprises a phospholipid bilayer. The membrane of the liposome can comprise a phospholipid bilayer. A micelle can comprise a phospholipid membrane. In these embodiments, the charge-modified globin is associated with the phospholipid membrane by being bound to the phospholipid membrane. The membrane may comprise lipids other than phospholipids, for example, cholesterol. The membrane may also comprise other components, such as integral membrane proteins. This may especially be the case where the membrane is a cell membrane.

In one embodiment, the charge-modified globin binds to the external solvent-exposed surface of the phospholipid membrane. This binding is preferably mediated by electrostatic forces. Typically, the external solvent-exposed surface of a phospholipid membrane comprises a negative charge, particularly in the case of a cell. However, it is also possible that the membrane can comprise a solvent-exposed surface comprising a positive charge.

To mediate electrostatic interactions with such membranes, the charge-modified globin can comprise an increased overall positive surface charge (i.e. a cationised globin) or an increased overall negative surface charge (i.e. an anionised globin).

A cell, liposome or micelle can have membranes comprising further molecules that can act as binding sites for charge-modified globins. For example, cells display various proteins, lipids and glycans on the exterior surface. Liposomes and micelles can be constructed to display various labels on their surface. In one embodiment, the charge-modified globin is associated with a molecule displayed on the exterior surface of the cell, liposome or micelle.

In one embodiment, the charge-modified globin is embedded in the membrane. The membranes of cells, and liposomes typically comprise at least one lipid bilayer, which has a hydrophobic interior bounded on each surface by hydrophilic functional groups. The charge-modified globin can be coated with a hydrophobic coating, as discussed in more detail below, which provides a hydrophobic charge-modified globin that can be embedded within the lipid bilayer. The term “embedded” indicates that the hydrophobic charge-modified globin is located at least partially within the phospholipid bilayer, or layer in the case of a micelle. That is, hydrophobic charge-modified globin at least partially intersects with the phospholipid bilayer or layer, rather than merely interacting with a surface of the phospholipid bilayer or layer.

In a preferred embodiment, the charge-modified globin is not internalised. In other words, the charge-modified globin remains in contact with the membrane and is not released into the cell or liposome interior.

In a preferred embodiment, the antitumour cell, antitumour liposome or antitumour micelle is an antitumour cell. ‘Antitumour cell’ can mean any cell that has antitumour properties. As such, the cell can be a natural cell, artificial cell, modified cell or cell organelle. The term ‘modified cell’ includes cells that have been modified in vitro, and cells that have been modified in vivo, for example by in vivo gene editing. In this specification, the term “cell” encompasses a protoplast or spheroplast, i.e., a cell normally comprising a cell membrane but having had at least some of said membrane removed or disrupted, for example, by a mechanical or enzymatic process. This may include, for example, a kit comprising a cell where at least some of said membrane has been removed or disrupted to assist with further modification of the cell, for example by transformation of the cell.

Typically, the cell is an animal cell such as a mammalian cell. The mammalian cell may be a human, mouse, dog, cat or horse cell, or a bovine, porcine or ovine cell. In a preferred embodiment, the cell is a human cell. Alternatively, the cell can be from a humanised animal, such as a humanised mouse. This is particularly preferred as human or humanised cells are favoured as they should be less immunogenic.

Typically, the cell is an immune cell that has cytotoxic properties that allow for killing of tumour cells. Preferably, the immune cell is a tumour-infiltrating cell, such as a lymphocyte, neutrophil, dendritic cell or macrophage. More preferably, the cell is a lymphocyte such as a cytotoxic T cell, natural killer T cell or natural killer cell. It is particularly preferred that the cell is a T cell, such as a CD3+ T cell. The CD3+ T cell is preferably of the CD4+ or CD8+ subtype, preferably the CD8+ subtype. In one embodiment, the T cell is a chimeric antigen receptor T (CAR-T) cell.

Importantly, it has been demonstrated that association of certain proteins (i.e. charge-modified GFP) with T cell membranes have toxic effects on T cells (FIG. 2). In contrast, a representative charge-modified globin (myoglobin) was shown to associate with the membrane of murine T cells with an unexpected reduced toxicity compared to GFP (FIG. 2b) and, even more surprisingly, with no observable toxicity to human Jurkat T cells (FIG. 2a).

Furthermore, the activity of a T cell is dependent on the structure and composition of the solvent-exposed outer surface of the T cell membrane, where a suite of proteins are required for recognition of ligands that mediate the immune response. The globins of the invention associate with this T cell membrane, but must do so without interfering with the T cell function. A surprising result, of critical importance, is that the globins can associate with the membrane of human Jurkat T cells without any appreciable loss of T cell activity (FIG. 3). This suggests that there is no interference, steric or otherwise, caused by the globins upon the interaction between the T cell receptors and their ligands.

Further surprising effects were seen with T cell behaviour in a normoxic environment as compared with behaviour in a hypoxic environment used as a representative model of a solid tumour environment. Both CD4+ and CD8+ subtypes of the murine T cell were examined. T cell proliferation was reduced upon association of myoglobin with the T cell membrane when exposed to a normoxic environment, but proliferation was restored in the hypoxic environment (FIGS. 4a and b). For the CD4+ subtype, a similar pattern was seen for activity. T cell activity was reduced for the T cell/myoglobin complex in a normoxic environment, but activity of the T cell/myoglobin complex was restored in the hypoxic environment (FIG. 4c). This unexpected result infers that these T cell/myoglobin complexes are particularly tuned to the hypoxic solid tumour environment. That is, the reduction of proliferation of T cell/myoglobin complexes in normoxic conditions can help to ensure globin is not diluted each time a T cell proliferates (i.e. by dilution during proliferation amongst the T cell progeny) generating T cell progeny with reduced globin concentrations. Likewise, activity of the T cell/myoglobin complexes in FIG. 4c is suppressed until the cells are in a hypoxic environment, making these cells particularly tuned to targeting and acting on hypoxic solid tumours. An even more surprising effect was observed for the CD8+ cell/myoglobin complex activity. In the hypoxic environment, T cell activity was actually significantly enhanced, making this a particularly exciting complex for antitumour therapy.

The charge-modified globin may be associated with the membrane of the cell for 1-30 days, or 1-15 or 1-10 or 1-5 days after the cell according to the invention is formed, by contacting the cell with the charge-modified globin. For example, the charge-modified globin may be associated for about 1, 2, 3, 4, 5, 6, 7, 8, 9 or about 10 days.

In one embodiment, the antitumour cell, liposome or micelle is an antitumour liposome or micelle. The liposome or micelle is typically a water-soluble liposome or micelle. The liposome or micelle will comprise components that give the liposome or micelle tumour-killing properties. In a preferred embodiment, the antitumour cell, liposome or micelle comprises a therapeutic agent such as a checkpoint inhibitor, immunotherapeutic or chemotherapeutic agent. Checkpoint inhibitors include but are not limited to peptides or proteins that bind to and block PD-1, PDL-1, and/or CTLA-4, for example peptides generated by phage display technologies to have high-affinity for and be inhibitory to PD-1, PDL-1 and/or CTLA-4, or antibodies or antibody fragments that are raised or designed to have high-affinity for and be inhibitory to PD-1, PDL-1 and/or CTLA-4. A number of checkpoint inhibitors are known in the art.

The liposome or micelle may also comprise targeting components, such as antibodies or other targeting proteins, that allow the liposome or micelle to specifically target tumour cells.

The globin can be a haemoglobin, myoglobin, neuroglobin, or cytoglobin. In a preferred embodiment, the globin is myoglobin. Globins reversibly bind and transport oxygen. As such, the globins bind oxygen and carry the oxygen until oxygen demand releases the oxygen. Myoglobin, in particular, is also known to act as a weak peroxidase and radical scavenger.

The globin can be linked to a secondary anti-cancer molecule. Alternatively, the globin can be linked to a reactive functional group for linking to a secondary antitumour molecule. For example, the reactive functional group may be one half of a bioconjugation system such as the SpyCatcher/SpyTag system (Reddington & Howarth (2015) Curr. Op. Chem. Biol. vol. 29 p 94-99; WO2014/176311), or streptavidin/biotin.

As such, the globin acts as an “anchor protein” that anchors the secondary antitumour molecule to the cell, liposome or micelle. This means that the charge-modified globin associated with the membrane of the cell, liposome or micelle provides at least two important advantages. The first advantage is the ability to readily deliver oxygen to both the tumour cell and the antitumour cell, liposome or micelle. The second advantage is the ability to anchor a secondary antitumour molecule to the membrane. As the charge-modified globin is associated with the membrane, the secondary molecule is advantageously presented on the exterior surface of the antitumour cell, liposome or micelle.

The secondary molecule may be a protein which is not a cationised or anionised protein. In cases where the charge-modified globin is embedded in the membrane, the secondary antitumour molecule can be positioned such that it is not embedded, as described in PCT/GB2018/052534.

The secondary antitumour molecule can be selected from any antitumour molecules known in the art. For example, the secondary antitumour molecule can be any one of an antibody, lectin, integrin or adhesion molecule. Specifically, the antitumour molecule can be any one of:

(1) A tumour cell binding molecule. Tumour cell binding molecules assist with targeting and sustained binding of tumour cells. The sustained binding can help ensure the antitumour cell, liposome or micelle is retained in the region of the tumour cell for a period of time that allows both the cell, liposome or micelle and the globin to have an enhanced effect on the tumour cell.

(2) A checkpoint inhibitor. Checkpoint inhibitors assist with reducing a tumour's ability to evade the immune system, complementary to the globin's effect on reducing hypoxia in a tumour, which also reduces the tumour's ability to evade antitumour therapies such as antitumour cells, liposomes and micelles.

(3) An enzyme that remodels the extracellular matrix of a tumour to enable enhanced penetration of immune cells into the tumour mass.

(4) An enzyme that metabolises tumour-associated compounds. For example, tumours have an increased rate of adenosine triphosphate metabolism, leading to excess adenosine in the tumour microenvironment. This stimulates adenosinergic receptors that lead to immunosuppressive effects. Presenting an enzyme, such as adenosine deaminase, at the immune cell surface would reduce this immunosuppressive signalling.

In one embodiment, the globin and the secondary anti-cancer molecule are contained within a fusion protein.

The globin is a charge-modified globin. The globin can be a cationised or anionised globin.

Cationised globin can be obtained by covalent bonding of a cationic or polycationic linker to an acidic amino acid side chain on the parent globin. For example, this may be achieved by mixing the protein with N,N′-dimethyl-1,3-propanediamine (DMPA) or an analogue thereof, in the presence of a carbodiimide such as N-(3-dimethylaminopropyl)-N′-ethylcarbodiimide hydrochloride (EDC) or dicyclohexyl carbodiimide (DCC). The reaction is shown below, which shows that acid residues (1) are activated towards nucleophilic attack by addition of the zero length cross-linker EDC (2) to form activated o-acylisourea groups (3). The nucleophile DMPA (4) then attacks the activated carbonyl and eliminates isourea to form cationised residues (5). Therefore, the cationised globin may comprise the linker —CH₂C(O)NCH₃(CH₂)₃N(CH₃)₂H⁺. DMPA or an analogue thereof may be added to the protein prior to mixing with EDC, to ensure the presence of an excess of DMPA or an analogue thereof and thereby avoid cross-linking of proteins to one another.

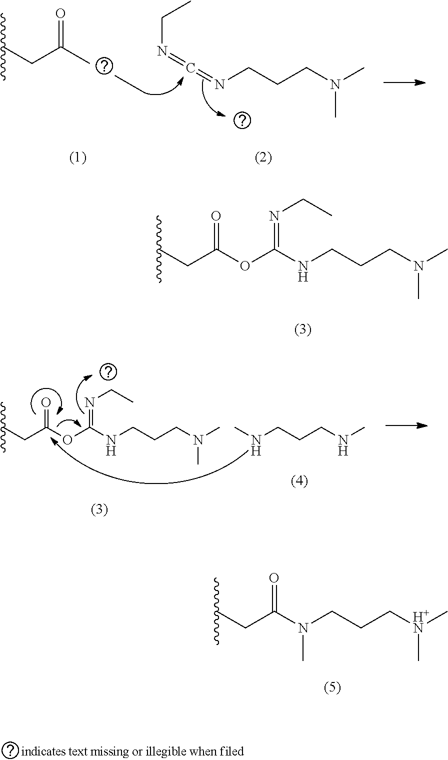

The step of covalent bonding of a cationic linker to an acidic amino acid side chain on the protein may be carried out in the presence of N-hydroxysuccinimide (NHS) or its water-soluble analogue Sulfo-NHS, to improve the stability of electrostatic coupling.

In the present invention, the mixing of the protein with DMPA or an analogue thereof in the presence of a carbodiimide may be allowed to continue for a limited time so as to avoid protein denaturation and/or aggregation. Such a limited time may be, for example, up to or for about 2 hours, or up to or for about 10, 15, 20, 25, 30, 35, 40, 45, 50, 55, 60, 65, 70, 75, 80, 85 or about 90 minutes. Alternatively or additionally, the product of the mixing with DMPA in the presence of a carbodiimide may be subjected to subsequent size exclusion chromatography, with the product from the chromatography being utilised as the cationised protein. The skilled person is capable of determining the theoretical size of the desired charge-modified globin, so as to determine the appropriate chromatography eluate to collect, for example by use of a calibrated chromatography column.

An analogue of DMPA may be N,N′-dimethylhexane-1,6-diamine (DMHA), N,N′-dimethylethylenediamine (DMEA), 3-dimethylamino propylamine (DMAPA), ethylenediamine (EN), 1,3-diaminopropane (DAP), 1,4-diaminobutane (DAB), 1,5-diaminopropane (DAP), 1,6-diaminohexane (DAH), hexamethylenediamine (HMA), 1,7-diaminheptane (DAH) 1,8-diaminooctane (DAO) and 2-(2-aminoethyl)guanidine (AEG).

Other suitable nucleophiles may be contemplated by the skilled person, for example, charged nucleophiles. For example, nucleophiles could also include other primary, secondary and tertiary alkyl diamines and alkyl diamines terminated with a quaternary amine if the opposing terminus contains either a primary, secondary or tertiary amine. Polyalkylamines such as polyethylenimine as either linear chains or branched structures are also contemplated.

Alternatively, the electrostatically modified protein may be obtained by anionisation of the protein. This may be achieved, for example, by nucleophilic addition of dicarboxylic acids (HOOC—R—COOH) to lysine side-chains of the native proteins.

In a further alternative, the charge-modified globin may be obtained by recombinant expression of a sequence having an altered charge with respect to the native globin, in particular a more positive or a more negative overall charge compared to the native globin. Recombinant modification may comprise recombinantly expressing charge-modified globin which is a mutant comprising one or more amino acid substitutions within its overall amino acid sequence compared to the sequence of the non-mutant globin, the amino acid substitutions introducing a different surface charge distribution to the charge-modified globin compared to the native globin, by providing a different amino acid charge to the native amino acid at the or each substitution position.

For example, an amino acid having an uncharged side group may be replaced by an amino acid having a positively or negatively charged side group (to give an overall charge change of +1 or −1 respectively), or an amino acid having a negatively charged side group may be replaced by an amino acid having a positively charged side group (to give an overall charge change of +2), or an amino acid having a positively charged side group may be replaced by an amino acid having a negatively charged side group (to give an overall charge change of −2), provided that the tertiary structure and/or biological activity of the protein is not significantly altered. This rational design approach may be especially advantageous if the function/activity of the protein depends on the involvement of a particular amino acid, for example one having a charged side group, since the user can direct globin surface charge alterations to non-critical amino acid positions; this may not always be possible with the chemical modification methods described elsewhere herein.

Typically, the amino acid sequence identity, determined at a global level (otherwise known as “global sequence identity”), between the native globin and the recombinantly modified charge-modified globin is at least about 60%, for example at least about 65%, 70%, 75%, 80%, 85%, 90%, 91%, 92%, 93%, 94%, 95%, 96%, 97%, 98% or at least about 99%. Determination of sequence identity at a global level may be carried out using, for example, the Needleman-Wunsch Global Sequence Alignment Tool available on the internet via the NCBI Blast® internet site (blast.ncbi.nlm.nih.gov/Blast.cgi). This tool allows a user to compare two sequences across their entire span. Where the globin is part of a fusion protein, the comparison is made with the globin part of the fusion protein.

Typically, the native globin consists of amino acids which are naturally occurring, for example which are selected from proteinogenic amino acids (including canonical amino acids). A proteinogenic amino acid is one which is incorporated into proteins by natural translation processes. Non-limiting examples of amino acids which may be included within the charge-modified globin are provided in Tables 1 and 2 below:

TABLE 1
examples of proteinogenic amino acids; bold indicates positively
charged amino acids, italic indicates negatively charges amino acids.

| | | | | |
|---|---|---|---|---|
| Alanine | Phenylalanine | Glutamine | Arginine | Selenocysteine |
| Isoleucine | Tryptophan | Serine | Histidine | Pyrrolysine |
| Leucine | Tyrosine | Threonine | Lysine | |
| Methionine | Asparagine | Aspartic acid | Glycine | |
| Valine | Cysteine | Glutamic acid | Proline | |

Modifications involving non-proteinogenic amino acids are also contemplated. Non-naturally occurring amino acids may also be included (such as those which may be introduced into a protein by use of a unique codon and a corresponding aminoacyl-tRNA system).

TABLE 2
examples of non-proteinogenic amino acids

| | | | | |
|---|---|---|---|---|
| β-alanine | γ-aminobutyric | δ-aminovulinic | 4-aminobenzoic | aminoisobutyric |
| | acid | acid | acid | acid |
| dehydroalanine | cystathione | lanthionine | djenkolic acid | diaminopimelic |
| | | | | acid |
| α-amino-n- | norvaline | norleucine | alloisoleucine | t-leucine |
| butyric acid | | | | |
| α-amino-n- | pipecolic acid | α,β- | α,γ- | ornithine |
| heptanoic acid | | diaminopropionic | diaminobutyric | |
| | | acid | acid | |
| allothreonine | homocysteine | homoserine | β-amino-n- | β- |
| | | | butyric acid | aminoisobutyric |
| | | | | acid |
| γ-aminobutyric | α- | isovaline | sarcosine | N-ethyl glycine |
| acid | aminoisobutyric | | | |
| | acid | | | |
| N-propyl | N-isopropyl | N-methyl glycine | N-ethyl glycine | N-ethyl alanine |
| glycine | glycine | | | |
| N-methyl β- | N-ethyl β-alanine | isoserine | α-hydroxy-γ- | homonorleucine |
| alanine | | | aminobutyric acid | |
| tellurocysteine | telluromethionine | citrulline | γ- | |
| | | | carboxyglutamate | |
| hydroxyproline | hypusine | pyroglutamic | | |
| | | acid | | |

In one embodiment, chemical cationisation or anionisation can be performed on recombinantly expressed charge-modified globin, in order to further modify the charge.

When using recombination techniques, the recombinant DNA sequence may encode for a fusion protein comprising the charge-modified globin and a proteinogenic secondary antitumour molecule, as described above.

The charge-modified globin can comprise a polymer surfactant coating, providing a polymer-coated charge-modified globin. This construct can comprise a charge-modified globin having one or more surfactant molecules electrostatically complexed to a charged amino acid residue at the surface of the protein. The preparation of similar constructs was described, for example, by Perriman et al. (2010; Nature Chem. vol. 2, 622-626), Brogan et al. (2013; J. Phys. Chem. B vol. 117, 8400-8407) and Sharma et al. (2013; Adv. Mater. vol. 25, 2005-2010). The conjugates are proteins having an amphiphilic surfactant corona, as described herein, around at least a portion of the overall structure. The presence of such a corona may be confirmed by comparison of the conjugate with the corresponding native globin, to detect changes in charge and/or size. Techniques such as mass spectrometry, zeta potentiometry, small angle X-ray scattering and/or dynamic light scattering, particularly a combination of two or more of these, may be employed to detect such changes.

The polymer-coated charge-modified globin may comprise a polyethylene glycol (PEG)-containing surfactant. For example, the surfactant may have the general structure of Formula I below:

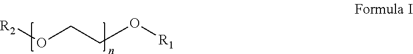

In Formula I, n can be any integer including or between 5 and 150, for example any integer including or between 8 and 110. For example, n may be 8, 9, 10, 11, 12, 13, 14, 15, 16, 17, 18, 19, 20, 25, 30, 35, 36, 37, 38, 39, 40, 45, 50, 55, 60, 65, 70, 75, 80, 85, 90, 95, 100, 105 or 110.

R₁ may be:

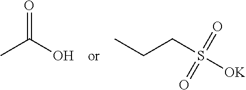

R₂ may be CₓH₍₂ₓ₊₁₎, where x is any integer including or between 8-18; for example, x=may be 11, 12 or 13. R₂ may also be an unsaturated hydrocarbon having 8-18 carbon atoms, for example 11, 12 or 13 carbon atoms. In a further alternative, R₂ may be:

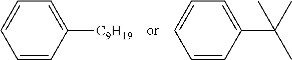

The surfactant may be one of those described herein, such as S621 (Sigma-Aldrich catalogue no. 463221), S907 (Sigma-Aldrich catalogue no. 463256), S1198 (Sigma-Aldrich catalogue no. 473197), or S1783 (oxidised form of glycolic acid ethoxylate 4-nonylphenyl ether, Sigma-Aldrich catalogue no. 238678).

These anionic surfactants have the following structures:

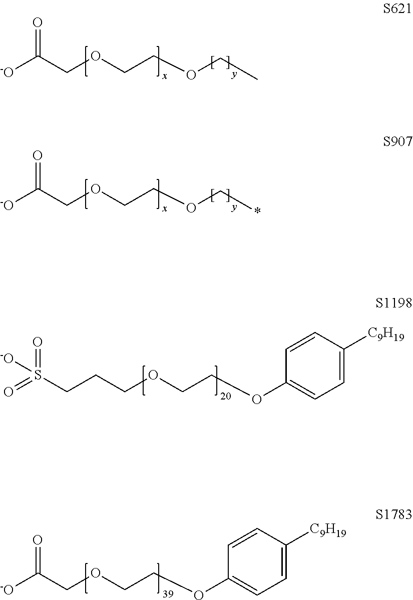

For S621 and S907 x=11-13

For S621, y=7-9

For S907, y=14-15

The molecular weight and polydispersity were measured by mass spectrometry and were found to be as follows:

TABLE 3
molecular weight and polydispersity of surfactants

| Name | MWt | PDi (Ð_{M}) |
|---|---|---|
| S621 | 621 | 1.05 |
| S907 | 907 | 1.06 |
| S1198 | 1198 | 1.03 |
| S1783 | 1997 | 1.12 |

The “polydispersity” reflects the fact that synthetic polymers produced from chemical reactions have a distribution of molecular masses arising from the intrinsically entropic process of polymerisation. The degree of variation is dependent on both the reaction mechanism and the reaction conditions. This degree of variation is defined by the dispersity (D), which was until recently known as the “polydispersity”. It is defined by the equation:

[symbol]

M=M_{w}/Mₙ

where M_{w} is the weight-average molar mass and Mₙ is the number-average molar mass.

The dispersity of a polymer can be estimated using matrix-assisted laser desorption ionization time-of-flight mass spectrometry (MALDI-TOF).

The protein-polymer surfactant conjugate may comprise a surfactant having a molecular weight of at least about 500 Da, for example, at least about 1000, 1100, 1200, 1300, 1400, 1500, 1600, 1700, 1800, 1900, 2000, 2200, 2400, 2600, 2800, 3000, 3200, 3400, 3600, 3800 or at least about 4000 Da.

The protein-polymer surfactant conjugate may comprise a surfactant which is 51783 (i.e., oxidised glycolic acid ethoxylate 4-nonylphenyl ether). Alternatively or additionally, the protein-polymer surfactant conjugate may comprise a cationic surfactant, for example, PEG-15 hydrogenated tallowmethylammonium chloride (sold as Ethoquad® HT25).

The cell, liposome or micelle according to the invention may be present within a complex composition further comprising at least one additional component, for example, water, a buffer solution, one or more components required to form a pharmaceutical composition as described below.

According to a second aspect, the invention provides a pharmaceutical composition comprising the antitumour cell, liposome or micelle according to the first aspect, further comprising a pharmaceutically acceptable carrier, diluent or vehicle.

According to a third aspect, the invention provides a cell, liposome or micelle according to the first aspect, or the pharmaceutical composition according to the second aspect, for use in the treatment of cancer.

The term “pharmaceutical composition” as referred to throughout this specification, may be a composition which comprises a pharmaceutically acceptable carrier, diluent or vehicle. For example, a pharmaceutical composition as referred to herein may be in the form of a sterile injectable preparation which may be an aqueous or an oleaginous suspension, or a suspension in a non-toxic parenterally-acceptable diluent or solvent. The aqueous suspension may be prepared in, for example, mannitol, water, Ringer's solution or isotonic sodium chloride solution. Alternatively, it may be prepared in phosphate buffered saline solution. The oleaginous suspension may be prepared in a synthetic monoglyceride, a synthetic diglyceride, a fatty acid or a natural pharmaceutically-acceptable oil. The fatty acid may be an oleic acid or an oleic acid glyceride derivative. The natural pharmaceutically-acceptable oil may be an olive oil, a castor oil, or a polyoxyethylated olive oil or castor oil. The oleaginous suspension may contain a long-chain alcohol diluent or dispersant, for example, conforming to Ph. Eur. and/or Ph. Helv. The pharmaceutical composition may comprise one or more pharmaceutically or otherwise biologically active agents in addition to the phospholipid composition of the invention. For example, the composition may include a therapeutic agent such as a conventional drug, antibody or other protein component

According to a fourth aspect, the invention provides a method of making the antitumour cell, liposome or micelle according to the first aspect, comprising a) providing a charge-modified globin; and b) contacting the antitumour cell, liposome or micelle with the globin.

In a method according to the fourth aspect of the invention when a cell is used, step (b) can involve incubating at a temperature of at least about 1° C. for a period of at least about 2 minutes, for example at a temperature of at least about 10° C. for a period of at least about 2 minutes. The temperature may typically be about 30-40° C., for example about 30, 31, 32, 33, 34, 35, 36, 37, 38, 39 or about 40° C., for example about 37° C.±about 1° C. Alternatively, certain cells, liposomes or micelles benefit from processing at lower temperatures such as 1-8° C., 2-7° C. or 3-6° C., for example about 1, 2, 3, 4, 5, 6, 7 or about 8° C. The time period may typically be 2-60 minutes, for example about 2, 3, 4, 5, 10, 15, 20, 30, 40, 50 or about 60 minutes, for example about 15, about 20 or about 30 minutes. The step may take place in an atmosphere of about 0-10% CO2, for example, of about 5% CO2. When a liposome or micelle is used, step (b) may be conducted at room temperature (e.g., between about 15° C. and about 25° C.) with <1% CO2, for example, in air.

Step (b) of the method according to the fourth aspect of the invention may optionally followed by a step (c) of washing the cell, liposome or micelle, for example using a buffer such as Phosphate Buffered Saline (PBS), for example with two or more washing steps. The skilled person is readily able to adapt such steps as required, and to determine when a washing step is desirable.

Step (a) can comprise providing a charge-modified globin and a polymer surfactant under conditions which enable electrostatic conjugation of the polymer surfactant with the globin. The surfactant may be added in solid or liquid form to a solution of the globin. The surfactant may be added in an amount equivalent to 0.5-5 moles surfactant per cationic site on the protein, for example, equivalent to about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9 or about 3.0 moles surfactant per cationic site on the protein. The protein may be in a solution with a suitable buffer such as a HEPES buffer, with or without CoCl₂, or in a Tris-HCl buffer. The selection of an appropriate buffer is within the routine abilities of the skilled person. The conditions may include a pH of between 5 and 8, for example of about 5, 6, 7 or about 8 (encompassing any individual intermediate pH value between 5.1 and 5.9, between 6.1 and 6.9, and between 7.1 and 7.9), and may include agitation of the mixture for 0-30 hours, for example, for about 1, 2, 3, 4, 5, 6, 7, 8, 9, 10, 11 or about 12 hours, at a temperature of 0-25° C., for example at about 4° C. or at about room temperature. For example, the conjugation conditions as described by Armstrong et al. (Nat. Commun. (2015) June 17; 6:7405) may be suitable.

A “cationic site” is a position within the amino acid sequence of the protein which has an amino acid with a positively charged side chain or comprising a cationic (i.e., positively charged) linker. The number of cationic sites within a globin may be determined without use of inventive skill by the skilled person.

The surfactant may comprise polyethylene glycol, which may, for example, have a molecular weight of at least about 500 Da, for example, at least about 1000, 1100, 1200, 1300, 1400, 1500, 1600, 1700, 1800, 1900, 2000, 2200, 2400, 2600, 2800, 3000, 3200, 3400, 3600, 3800 or at least about 4000 Da. The surfactant may be in a buffer solution at a concentration of 5-50 mg/mL, for example, about 10, 15, 20, 25, or about 30 mg/mL.

The surfactant may be 51783 (i.e., oxidised glycolic acid ethoxylate 4-nonylphenyl ether). Alternatively, the surfactant conjugate may comprise a cationic surfactant, for example, PEG-15 hydrogenated tallowmodium chloride (sold as Ethoquad® HT25).

The charge-modified globin may be linked to a secondary antitumour molecule, as described above, prior to contacting with the surfactant.

Step (a) may also comprise, prior to contacting the cell, liposome or micelle with the globin, a buffer exchange step. The buffer exchange step may comprise a spin concentration of the product of the step of contacting the cationised or anionised protein with the surfactant. Alternatively, the buffer exchange step may comprise a dialysis step. Such methods are within the routine ability of the skilled person.

The charge-modified globin can be generated by chemically modifying the charges on a native globin. For example, at least one acidic amino acid side chain may comprise a —CH₂C(O)NCH₃(CH₂)₃N(CH₃)₂H⁺ linker. This may be achieved by a method in which a solution of N,N′-dimethyl-1,3-propanediamine (DMPA) or an analogue thereof is mixed with a native globin (as defined above), in the presence of N-(3-dimethylaminopropyl)-N′-ethylcarbodiimide hydrochloride (EDC). An analogue of DMPA may be N,N′-dimethylhexane-1,6-diamine (DMHA), N,N′-dimethylethylenediamine (DMEA), 3-dimethylamino propylamine (DMAPA), ethylenediamine (EN), 1,3-diaminopropane (DAP), 1,4-diaminobutane (DAB), 1,5-diaminopropane (DAP), 1,6-diaminohexane (DAH), hexamethylenediamine (HMA), 1,7-diaminoheptane (DAH) 1,8-diaminooctane (DAO) and 2-(2-aminoethyl)guanidine (AEG). Other suitable nucleophiles may be contemplated by the skilled person, for example, charged nucleophiles. For example, nucleophiles could also include other primary, secondary and tertiary alkyl diamines and alkyl diamines terminated with a quaternary amine if the opposing terminus contains either a primary, secondary or tertiary amine. Polyalkylamines such as polyethylenimine as either linear chains or branched structures are also contemplated.

Therefore, a globin (i.e. a charge-modified globin precursor) can be converted to the charge-modified globin by a method comprising:

i) mixing a solution of globin with a pH-neutralised solution of N,N′-dimethyl-1,3-propanediamine (DMPA) or analogue thereof and optionally adjusting the mixture to pH 5-7;

ii) subsequently or concurrently adding a carbodiimide such as N-(3-dimethylaminopropyl)-N′ethylcarbodiimide hydrochloride (EDC) and adjusting the mixture to pH 4-7;

iii) agitating the mixture from (ii) for 1-30 hours at pH 4-7, at a temperature of 0-25° C.;

iv) dialysing the protein in the mixture from (iii) against water or buffer for at least 4 hours at pH 6.5-8.5;

v) if necessary, adjusting the pH of the mixture from (iv) to pH 6.5-8.5.

In the method, either step (iii) continues for no longer than about 120 minutes, for example, for no longer than about 90 minutes; and/or the method further comprises a step (vi) of conducting size exclusion chromatography on the mixture from step (iv), or from step (v) when present, and obtaining an eluate comprising a charge-modified globin at the required molecular weight. Either or both of these limitations ensure that the process is controlled to reduce or prevent protein denaturation and/or aggregation.

The solution of native globin used in step (i) may be prepared in any conventional buffer, for example, HEPES. The native globin is mixed with DMPA at a ratio of moles DMPA:number of anionic sites on the protein of 100:1-400:1, for example, about 100:1, 150:1, 200:1, 250:1, or about 300:1. EDC is added at a ratio of moles EDC:number of anionic sites on the protein of 30:1-60:1, for example, about 30:1, 31:1, 32:1, 33:1, 34:1, 35:1, 40:1, 45:1 or about 50:1.

An “anionic site” is a position within the amino acid sequence of the globin which has an amino acid with a negatively charged side chain. The number of anionic sites within a globin may be determined using the routine ability of the skilled person.

Step (ii) may be completed at the same time as step (i), i.e. the protein solution, DMPA and EDC may be mixed concurrently. Where step (ii) is completed after step (i), step (ii) may be a single step as defined above and immediately followed by step (iii), or may be subdivided into two steps (iia) in which a portion of the EDC is added to the mixture from step (i) and the mixture agitated for about 2, 3, 4, 5, 6, 7 or about 8 hours at a temperature of 0-25° C., followed by (iib) in which further EDC is added to the mixture from (iia) and the agitation continues; step (iib) is followed by step (iii).

The required agitation in step (iii) may be achieved by any conventional means such as stirring, for example, and the pH may be about 4, about 5, about 6 or about 7 (encompassing any intermediate pH value between 4.1 and 4.9 and between 5.1 and 5.9 and between 6.1 and 6.9). When the time period in step (iii) exceeds 120 minutes, it may continue for about 20-30 hours, for example, about 20, 21, 22, 23, 24, 25, 26, 27, 28, 29 or about 30 hours, for example about 24 hours. All steps may, for example, be conducted at about room temperature, for example, 18-23° C., or may be conducted at about 4° C.

The skilled person may determine the appropriate time period for step (iii), whether or not there is a subsequent size exclusion chromatography step, by conducting the step (iii) for a range of time periods and testing for the retention of globin activity, to determine the optimal time period for step (iii).

In an alternative general method, the polymer surfactant-coated charge-modified globin may be prepared by contacting a charge-modified globin which is an anionised globin as described with a surfactant which is a cationic surfactant. For example, the globin may be anionised by nucleophilic addition of dicarboxylic acids (HOOC—R—COOH) to the lysine side-chains of the native protein.

Alternatively or additionally to the above modifications, the charge-modified globin can be obtained by a method comprising expression of a recombinant DNA sequence encoding for the charge-modified globin. For example, the charge-modified globin may be obtained by a method comprising expression of a recombinant DNA sequence encoding for a charge-modified protein. The resulting protein, which is the charge-modified globin, subsequently may be isolated.

For example, preparation of a charge-modified globin may involve substituting an amino acid having an uncharged side group with an amino acid having a charged side group, or substituting an amino acid with a charged side group with a side group having the opposite charge, provided that the tertiary structure and/or biological activity of the protein is not significantly altered. This may be especially advantageous if the function/activity of the protein depends on the involvement of an amino acid with a charged side group, since the user can direct protein surface charge alterations to non-critical amino acid positions.

Typically, the amino acid sequence identity, determined at a global level (otherwise known as “global sequence identity”), between the recombinantly modified protein (i.e., the charge-modified globin) and the native protein is at least about 60%, for example at least about 65%, 70%, 75%, 80%, 85%, 90%, 91%, 92%, 93%, 94%, 95%, 96%, 97%, 98% or at least about 99%. Determination of sequence identity at a global level may be carried out using, for example, the Needleman-Wunsch Global Sequence Alignment Tool available on the internet via the NCBI Blast® internet site (http://blast.ncbi.nlm.nih.gov/Blast.cgi). As mentioned above, the sequence identity of a functionally important domain may be at least about 90%, 91%, 92%, 93%, 94%, 95%, 96%, 97%, 98% or at least about 99% identical between the charge-modified globin and the native globin.

The recombinant DNA sequence may be expressed according to any routine method, for example, using any expression system such as expression in E. coli, in accordance with the routine abilities of the skilled person. Isolation of the expressed anchor protein from the expression system is also within the routine abilities of the skilled person.

According to a fifth aspect, the invention provides a method of treating cancer, comprising administration of the cell, liposome or micelle according to the first aspect, or the pharmaceutical composition according to the second aspect, to a patient in need thereof.

In a preferred embodiment, the cancer of the third or fifth aspects of the invention is a solid tumour cancer. In particular, the cancer can be selected from: breast, colorectal, prostate, lung, stomach, liver, oesophageal, cervical, or pancreatic cancer. These are the most frequently-occurring solid tumour cancers. More specifically, the cancer can be selected from ICD-10 Version:2016 (international classification of disease, 10th Revision, published by the World Health Organisation, icd.who.int/browse10/2016/en) codes C00-C75.9.

According to a sixth aspect, the invention provides a charge-modified globin comprising any of SEQ ID NOs: 1-12 or a functional variant of any of these having at least about 60% sequence identity with any of SEQ ID NOs: 1-12. For example, the charge-modified globin may have at least about 65%, for example at least about 70%, 75%, 80%, 85%, 90%, 91%, 92%, 93%, 94%, 95%, 96%, 97%, 98% or at least about 99% sequence identity with any of SEQ ID NOs: 1-12. The charge-modified globin may optionally form part of a larger construct, such as a fusion protein.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires; in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows the results of a fluorescence activated cell sorting (FACS) analysis applied to human Jurkat T cells (a and c) or murine T cells (b and d) that have undergone labelling procedures with no protein, native myoglobin (nMyo), cationised myoglobin (Cat Myo), conjugated cationised myoglobin (Con Myo), supercharged GFP (scGFP) and conjugated supercharged GFP (Con scGFP). Jurkat T cells were gated on their expression of CD3 and then the percentage of cells staining for surface presentation of His-Tag present on the myoglobin and GFP (a) or GFP fluorescence (c). Murine T cells were gated on their expression of CD8 and the percentage of cells staining for surface presentation of His-Tag present on the myoglobin and GFP (b) or GFP fluorescence (d). All data are presented as representative data plot (inset) and by chart (showing mean %+/−SD, n=4).

FIG. 2 shows the results of a FACS analysis applied to human Jurkat T cells (a) and murine T cells (b) (that have undergone labelling procedures as described for FIG. 1) that were stained with Aqua cell viability dye, which were gated on their expression of CD3 (a) or CD8 (b) and the percentage of Aqua negative live cells was calculated as mean %+/−SD, n=4; representative data plot in inset, processed data shown by chart.

FIG. 3 shows the results of a FACS analysis applied to T cell receptor transgenic human Jurkat T cells (a and b) that have undergone labelling procedures with (a) no protein, native myoglobin, cationised myoglobin, and conjugated cationised myoglobin, or (b) no protein, scGFP, and conjugated scGFP, that were analysed for the upregulation of CD69 in response to C1R antigen presenting cells pulsed with 0 μM, 1 μM or 10 μM cognate peptide.

FIG. 4 shows the results of murine T cells (that have undergone labelling procedures as described for FIG. 1) stained with cell trace violet (CTV) and then activated for 4 days in the presence of anti-CD3, anti-CD28 and IL-2 under normoxic (squares) or hypoxic (circles) conditions (a and b), and the results of murine T cells activated under normoxic (squares) or hypoxic (circles) conditions tested for expression of high levels of CD44 (c and d), separated by T cells of the CD4 expressing subtype (a and c) and the CD8 expressing subtype (b and d), presented as representative plot (inset) and by chart (showing mean %+/−SD, n=4).

FIG. 5 shows (a) the labelling efficiency of surfactant-conjugated supercharged myoglobin (Myo14[S]) on Jurkat T-cells, the viability of Myo14[S]-coated (b) Jurkat T-cells and (c) activated CD8+ murine T-cells, (d) the cell counts of murine T-cells 3 and 5 days post-coating versus an uncoated control (U/T), (e) the percentage of divided CD8+ murine T-cells 3 and 5 days post-coating, and (f) the level of CD69 activation of coated Jurkat T-cells versus an uncoated control (Untreated).

FIG. 6 shows (a) the levels of exhaustion markers on surfactant-conjugated supercharged myoglobin (Myo14[S])-coated CD8+ murine T-cells versus uncoated controls (U/T), (b) the change in fluorescence over time of a hypoxia-sensitive dye at 0.5% 02 for Myo14[S]-coated and uncoated Jurkat T-cells (untreated), and (c) the viability and levels of markers of inflammation of human mesenchymal stem cells with or without Myo14[S] and interferon gamma.

### EXAMPLES

Materials and Methods

Myo14 Expression

Myo14 was obtained by expression in BL21(DE3) E. coli cells transformed with a plasmid containing the appropriate Myo14 gene by electroporation using routine methods. Briefly, a single colony was picked and placed in 10 mL of LB media for a starter culture, and incubated overnight at 37° C., shaken at 180 RPM. The starter culture was then used to inoculate 1 L of TB media supplemented with 0.02% glucose and 50 mg·L⁻¹ carbenicillin in a 2.5 L culture flask. The culture flask is then incubated at 37° C., 200 RPM. Once the TB reaches an optical density at 600 nm of 0.7-1.0, protein expression is induced with 1 mM IPTG. After 4 hours, the expression cultures are centrifuged at 4500×g for 20 minutes at 4° C. to pellet the cells. The supernatant is discarded, and the pellet can be frozen for storage or used immediately.

Myo14 Purification

Lysis buffer containing 20 mM HEPES, 1 M NaCl, at pH 7.0 was added to the Myo14 pellets, lysed using pulse sonication, and clarified in a centrifuge, using routine methods. The Myo14 is then purified using maltose binding protein affinity chromatography, and the maltose binding protein is then cleaved with TEV protease overnight at room temperature in a fully anoxic environment. Finally the resulting cleaved product is polished with size exclusion chromatography, using routine methods.

Preparation of Constructs

The chemically charge-modified myoglobin, termed cationised myoglobin, was prepared by covalent modification of acid residues with N,N-dimethyl-1,3-propanediamine (DMPA) via a 1-ethyl-3-(3-dimethylaminopropyl)carbodiimide hydrochloride (EDC) mediated reaction. A myoglobin solution in 2-(N-morpholino)ethanesulfonic acid (MES) buffer was added to a pH-neutralised solution of DMPA at a ratio of 300 moles DMPA per mole of acidic residues on the myoglobin. The solution was pH adjusted to pH 5.5-6.0. EDC was then added at a ratio of 50 moles EDC per mole of acidic residues on the myoglobin, and left to stir at 4° C. overnight. The resulting solution was then desalted to remove by-products via desalting columns, spin-concentrating and diluting into fresh buffer to achieve a minimum 1 million-fold dilution, or dialysing against fresh buffer.

Surfactant-conjugated cationised-myoglobin, termed conjugated myoglobin, was prepared by adding anionic surfactant to a solution of cationised myoglobin. The surfactant was an oxidised form of glycolic acid ethoxylate 4-nonylphenyl ether (51783, as described above), and was added at a ratio of 1 mole of surfactant per mole of positively charged residues on the myoglobin. The surfactant may be added in solid form, or pre-dissolved in an appropriate buffer.

The recombinantly charge-modified (“supercharged”) myoglobins were prepared by altering the amino acid sequence of myoglobin, before ordering the corresponding DNA from commercial sources. The 1st mutant (SEQ ID NO 1) was modified to incorporate lysine residues at positions that were found by proteomic analysis to be modified by the addition of DMPA in the cationised myoglobin. The Max mutant (SEQ ID NO 2) was modified to swap all acid residues for lysine residues. The polarised mutant (SEQ ID NO 3) was modified to incorporate lysine residues at the surface of the myoglobin except within a 10 A radius of the C-terminus in which positive residues were modified to glutamic acids. The ROSETTA mutant (SEQ ID NO 4) was modified to incorporate mutations suggested by the ROSETTA algorithm on the ROSIE web service (rosie.graylab.jhu.edu). The AVNAPSA mutant (SEQ ID NO 5) was modified to incorporate mutations suggested by the AVNAPSA algorithm on the ROSIE web service. The TaB mutant (SEQ ID NO 6) was modified to incorporate either lysine or arginine residues at surface accessible positions without hydrogen bonds or electrostatic interactions and with a B-factor above the mean B-factor plus one standard deviation from the crystal structure of PDBID 3RGK. The consensus B model (SEQ ID NO 7) was designed at the TaB mutant, but excluded mutations to residues that were not found to be mutated in any homologues. Myo7 (SEQ ID NO 8) was modified to include positively charged residues found in several homologues from closely-related species, and was used as a base for the remaining sequences. Myo14 (SEQ ID NO 9) was modified to include residues determined to be surface accessible by structure inspection. cMyo9 (SEQ ID NO 10), cMyo14 (SEQ ID NO 11) and cMyo15 (SEQ ID NO 12) were modified to include successively more mutations than Myo7 (SEQ ID NO 8) that were either lower frequency and/or from more distantly related species.

TCR transgenic Jurkat T cells (jTc) or CD3+ T cells purified from murine spleen and lymph nodes (mTc) were incubated for 20 mins with cationised, conjugated or native myoglobin or GFP or conjugated super charged GFP. Cells were washed three times and analysed, in three different experiments, for viability, activation and proliferation.

To test T cells with Myo14[S] TCR transgenic Jurkat T cells (jTc) or CD3+ T cells were used, purified from murine spleen and lymph nodes (mTc). T cells were incubated for 30 mins at 37° C. with Myo14[S] or untreated (U/T). Cells were washed two times and analysed for Myo14[S] coating, viability, phenotype, activation, resistance to hypoxia and proliferation.

Conjugate Coating and Viability Assay

jTc were harvested from culture and washed once with PBS then incubated in PBS with no protein or 3 μM conjugated super charged GFP (con-scGFP), 3 μM super charged GFP (scGFP), 10 μM of native myoglobin (nMyo), 10 μM of cationised myoglobin (Cat Myo) or 10 μM conjugated myoglobin (Con Myo) for 20 mins at 37° C. Coated jTc were washed three times in PBS and resuspended in PBS at 1×10⁶ jTc in 100 μl per well and stained with αCD3-AF594 (Biolegend, Cat No. 300446), αHIS-TAG-APC (Biolegend, Cat No. 362605) and Aqua (Invitrogen, Cat No. L34957) for 30 min at 4° C. Stained cells were washed twice with PBS and analysed using a Fortessa X20 Cytometer.

Whole T cells (mTc) were purified from the spleen and lymph nodes of one Balb-c female mouse using a Dyna-bead negative selection kit (ThermoFisher. Cat No. 11413D) as per the manufacturer's instructions. mTc were washed once with PBS then incubated in PBS with no protein or 3 μM conjugated super charged GFP (Con scGFP), 3 μM super charged GFP (scGFP), 10 μM of native myoglobin (nMyo), 10 μM of cationised myoglobin (Cat Myo) or 10 μM conjugated myoglobin (Con Myo) for 20 mins at 37° C. Coated mTc were washed three times in PBS and resuspended in PBS at 1×10⁶ mTc in 100 μl per well and stained with αCD3-AF700 (Biolegend, Cat No. 152316), αCD4-BV785 (Biolegend, Cat No. 100552), αCD8-APCef780 (Invitrogen, Cat No. 47008182), αHIS-TAG-APC (Biolegend, Cat No. 362605) and Aqua (Invitrogen, Cat No. L34957) for 30 min at 4° C. Stained cells were washed twice with PBS and analysed using a Fortessa X20 Cytometer.

Jurkat T Cell Activation Assay

TCR transgenic Jurkat T cells (jTc) are Jurkat T cells transduced with a T cell receptor (TCR) that recognises a cognate peptide when presented by MHC Class I HLA-A2. When stimulated by their cognate peptide the expression of CD69 increases and can be detected by FACS as a surrogate marker for T cell activation.

jTc and C1R were maintained in RPMI 1640 supplemented with 10% v/v FCS, 2 μM glutamine, 50IU/ml Penicillin, 50 μg/ml Streptomycin, 50 μM 2-β-mercaptoethanol and 10 mM HEPES (R10) at 37° C.

C1R-HLA-A2 were washed twice with PBS and then resuspended in PBS with either 0 μM, 1 μM or 10 μM cognate peptide for 50 mins at 37° C. Cells were washed twice in R10 and plated at 1×10⁵ cells in 100 μl per well in a 96 U-bottomed plate.

jTc were harvested from culture and washed once with PBS then incubated in PBS with no protein or 3 μM conjugated super charged GFP (con-scGFP), 3 μM super charged GFP (scGFP), 10 μM of native myoglobin (nMyo), 10 μM of cationised myoglobin (Cat Myo) or 10 μM conjugated myoglobin (Con Myo) for 20 mins at 37° C. Coated jTc were washed once in PBS and resuspended in R10 at 5×10⁴ jTc in 100 μl per well and were mixed with C1R cells.

Identical plates with coated jTc and peptide pulsed C1R cells were co-cultured for 6 hours at 37° C. in incubators with environmental oxygen (21%) levels or hypoxic oxygen level (5%).

After 6 hours plates were washed once with PBS, cells were then incubated with αCD69-APC (Biolegend, Cat No. 310910), αCD3-AF594 (Biolegend, Cat No. 300446) and the live cell stain, Aqua (Invitrogen, Cat No. L34957) at 4° C. for 30 mins. Cells were then washed twice with PBS and fixed with 2% Paraformaldehyde (PFA) overnight. Cells were washed twice in PBS to remove the fixative and were run on the High throughput module of a Fortessa X20 Cytometer.

Murine T cell Proliferation Assay

96 U bottomed plates were coated with 1 μg/ml αCD3 and 5 μg/ml αCD28 diluted in PBS for 1 hour at 37° C. PBS was removed.

Whole T cells (Tc) were purified from the spleen and lymph nodes of one Balb-c female mouse using a Dyna-bead negative selection kit (ThermoFisher. Cat No. 11413D) as per the manufacturer's instructions.

Purified Tc were washed once with PBS then incubated in PBS with no protein or 3 μM conjugated super charged GFP (Con scGFP), 3 μM super charged GFP (scGFP), 10 μM of native myoglobin (nMyo), 10 μM of cationised myoglobin (Cat Myo) or 10 μM conjugated myoglobin (Con Myo) for 20 mins at 37° C.

Coated Tc were washed twice in PBS and resuspended in PBS with 10 μM Cell Trace Violet (ThermoFisher. Cat No. C34557) at RT for 20 mins. CTV stained cells were washed in R10 and transferred at 4×10⁵ Tc per well in 200 μl R10 with 20 U/ml IL-2 to αCD3 and αCD28 pre-coated plates.

Identical plates with coated Tc were cultured for 4 days at 37° C. in incubators with environmental oxygen (21%) levels or hypoxic oxygen levels (5%).

After 4 days, plates were centrifuged at 500×g for 5 mins and washed once with PBS, cells were then incubated with two combinations of antibodies at 4° C. for 45 mins.

Combination 1: αCD3-AF700 (Biolegend, Cat No. 152316), αCD4-BV785 (Biolegend, Cat No. 100552), αCD8-APCef780 (Invitrogen, Cat No. 47008182), αCD44-BV605 (Biolegend, Cat No. 103047), αHIS-TAG-APC (Biolegend, Cat No. 362605) and Aqua (Invitrogen, Cat No. L34957).

Combination 2: αCD3-AF700, αCD4-BV785, αCD8-APCef780, αPDL-1-PE (Invitrogen, Cat No. 125982-82), Isotype Control-BV605 (Biolegend, Cat No. 400434), Isotype Control-APC (Invitrogen, Cat No. 17432181) and Aqua.

Cells were then washed twice with PBS and fixed with 2% Paraformaldehyde (PFA; FisherScientific, Cat No. 11586711) for 1 hr at 4° C. Cells were washed twice in PBS to remove the fixative and were run on the High throughput module of a Fortessa X20 Cytometer.

Tc were maintained in RPMI 1640 (ThermoFisher Scientific, Cat No. 21875-034) supplemented with 10% v/v FCS, 2 μM glutamine, 50 IU/ml Penicillin, 50 μg/ml Streptomycin, 50 μM 2-β-mercaptoethanol and 10 mM HEPES (R10) at 37° C.

Labelling Efficiency

Myo14 was labelled with FITC as per the manufacturer's instructions prior to conjugation. jTc were untreated or coated with 14 μM Myo14[S]-FITC and stained with αCD3-AF594 then analysed by flow cytometry. The mean proportion of FITC+CD3+Tc is presented with representative FACS plots (+/−SD, n=3).

FACS-Based Cell Viability Analysis

Live jTc coated with varying concentrations of Myo14[S] were stained for Aqua Live/dead cell stain and CD3-AF594. Tc were gated on Singlets and CD3+ Tc and then the mean percentage of live cells was calculated. Naive murine Tc were purified and activated in vitro with αCD3/CD28 and IL-2 for 1 day. On day 1 activated Tc were harvested and coated with Myo14[S] at varying concentrations. Coated, activated Tc were added back to culture with αCD3/CD28 and IL-2 for 1 day. Cells were harvested and stained with anti-CD8 and Aqua Live dead cell stain. Cells were gated on Singlets and CD8+ cells then the mean percentage of live cells was calculated.

FACS-Based Cell Proliferation Analysis

Whole Tc were purified from spleen and lymph nodes of one Balb-c mouse. Tc were untreated or coated with 2.5 μM Myo14[S]. Tc were activated with αCD3/CD28. Activated Tc were harvested at day 3 and day 5 and stained with trypan blue to exclude dead cells then counted. The mean cell number (+/−SD, n=3) is presented.

Purified whole Tc were stained with Cell Trace Violet then left untreated (U/T) or coated with 2.5 μM Myo14[S]. Tc were activated with αCD3/CD28 and IL2. Activated mTc were harvested at day 3 and day 5 and stained with αCD4 and αCD8. Cell were analysed by flow cytometry. Cells were gated on CD8 cells and the mean of all CTV diluted cells were included as ‘Divided CD8 Tc’ (+/−SD, n=3).

Antigen-Specific Activation

TCR transgenic jTc were untreated (U/T) or coated with 10 μM [Myo14-MBP][S]. jTc were cultured for 6 hours with C1R-HLA-A2+ cells peptide pulsed with the stated concentrations of cognate peptide. jTc were stained with αCD3-AF594, αCD69-APC and Aqua Live/Dead stain, the mean MFI of the activation marker CD69 was calculated on live, CD3+ jTc (+/−SD, n=3).

Hypoxia-Dye Assay

Image-iT Green hypoxia reagent (Thermo Fisher) was diluted to 5 mM by adding DMSO and mixing well. This stock was then added to jTc to a final concentration of 101 μM in R10 media, and incubated at 37° C., 20% O₂, 5% CO₂ for 40 minutes. The cells were then centrifuged at 500×g for 5 minutes, the supernatant poured away and replaced with fresh growth medium. The cells are then plated in a 96-well plate at 5×10⁵ cell per well and read on a fluorescence plate reader over time with the oxygen concentration fixed at the desired value, for example 0.5% 02.

hMSC Inflammation Assay

Human mesenchymal stem cells, isolated from four patients, were untreated or coated with 2.5 μM Myo14[S] and cultured for 7 days. As a positive control, cells were cultured with IFNγ. At day 7 cells were harvested, counted and stained for Aqua Live/Dead stain and the lineage markers, CD105 and CD73 and the inflammatory markers HLA-DR, HLA-ABC and PDL1. Cells were gated on singlets, live cells and CD105+CD73+ cells, then the mean percentage of cells expressing each marker was calculated.

2D In Vitro Tumour Killing Assays

Antigen-specific killing of tumour cells, such as IGR-Heu lung carcinoma cells, SKBR3, BT20, MCF7 breast cancer cells, SKOV3 ovarian cancer cells or HeLa-CD19 cells, are carried out using human cytotoxic T cells derived from either PBMCs or isolation of tumour-infiltrating lymphocytes, or CAR-T cells.

Upon isolation from PBMCs, T cells are activated using stimulating agents, such as irradiated autologous tumour cells, HLA-A2-binding HER-2/neu p 369-377 peptide or CD3/CD28 Dynabeads, and growth factors, such as IL-2.

Prior to co-culture with tumour target cells, T cells are coated with Myo14 or Myo14[S]. Uncoated T cells are used as a control for baseline killing activity. Tumour cell lines are either transfected for the expression of a reporter gene or incubated with dyes such as CFSE to be fluorescently labelled. During co-culture of activated T cells and tumour target cells, decrease in fluorescence is monitored via imaging, FACS or by using a plate reader to determine rate of tumour killing.

As positive control for tumour killing, an anti-cancer drug (i.e. staurosporine) is added to culture media on tumour cells alone.

3D In Vitro Tumour Killing Assays

Target tumour cells are transfected to express GFP and grown into 3D spheroids. Isolated T cells are added to tumour spheroids, after being coated with Myo14 or Myo14[S]. Uncoated T cells are used as a control for baseline killing activity. During co-culture, decrease in fluorescence is monitored via imaging to determine rate of tumour spheroid cell death.

To monitor T cell infiltration in tumour spheroids, T cells are fluorescently labelled with a different dye and co-cultures are imaged.

As positive control for tumour killing, an anti-cancer drug (i.e. staurosporine) is added to culture media on tumour spheroids alone.

In Vivo Tumour Killing Assays

Myo14 based constructs are tested in vitro and in vivo in multiple models to further demonstrate functionality and efficacy. For in vivo efficacy, and to demonstrate that Myo14 based constructs are suitable for multiple oncological applications, Myo14[S] and [Myo14-PD1][S] are coated onto Chimeric Antigen Receptor T cells (CAR-T) or transgenic Clone 4 T cells and adoptively transferred into mice bearing MDA-MB-231 human breast cancer tumour or RencaHA tumours, respectively. Each experiment is described in more detail below.

For the CAR-T/MDA-MB-231 experiments, 32 NSG mice are inoculated with subcutaneous MDA-MB-231. When the tumour reaches the pre-determined time point the mice are split into 4 groups. Group 1 is left untreated, Group 2 is treated with i.v. CAR-T, Group 3 is treated with i.v. Myo14[S] coated CAR-T and Group 4 is treated with i.v. [Myo14-PD1][S] coated CAR-T. The experiment is repeated at least once.

Tumour size is measured regularly with callipers as well as bioluminescent imaging of luciferase transduced MDA-MB-231 tumour cells. Flow cytometric analysis of serial tail bleeds is performed to determine the persistence and expansion of infused Tc.

Sections from treated tumour tissues are analysed by multiplex staining (multicolour Vectra Automated Quantitative Pathology Imaging System and quantitatively analysed using Definiens software) available through the Cancer Research UK Birmingham Cancer Centre. Tumours are stained with the following antibody panel:

Anti-human CD3, pan-cytokeratin (tumour marker), CD34 (CAR marker), carbonic anhydrase IX (hypoxia marker), human annexin V (apoptosis marker) and human IFN gamma (functional marker) human PD1 and human PDL1 (plus DAPI nuclear stain). This enables visualisation, analysis, quantification and phenotyping of immune cells and identification of cell-to-cell interactions within a single tumour tissue section.

Secondary lymphoid tissues and tumour are analysed by FACS for the presence of CAR-T cells. The phenotype of adoptively transferred CAR-T cells is analysed for surface expression of PD-1, CD45 and CD62L and intracellular cytokine staining for IFNγ and TNFα. The phenotype of treated tumour cells is analysed by surface expression of PDL-1, MHC Class I and MHC Class II.

In an additional in vivo tumour model, Myo14[S] and [Myo14-PD1][S] are coated onto transgenic Clone 4 T cells purified from 6-8 week old CL4 TCR-transgenic Thy1.1+ BALB/c mice. 20×106 CL4 TCR-Transgenic Tc are injected i.v. to 8 randomly selected female Balti-c mice that have been previously injected s.c. with 1×10⁶ RencaHa tumour cells. The growth of the RencaHa tumour is measured in 4 groups which are untreated, treated with uncoated Tc, treated with Myo14[S] coated Tc and [Myo14-PD1][S] coated Tc. The scientific and humane end point of the experiment is set at 27 days based on previous work. Tumour diameter in two dimensions is measured every other day using callipers and the rate of tumour progression and the final tumour volume calculated. Flow cytometric analysis of serial tail bleeds is performed to determine the persistence and expansion of infused Tc.

Adoptively transferred Tc with the Thy1.1 congenic marker are identified in tumours, spleen, tumour draining and tumour non-draining lymph nodes, lungs, brain and liver by fluorescent immuno-histochemistry to enumerate the distribution and tumour specific migration and retention of treated and untreated Tc. Furthermore, sections from tumour samples are stained with markers for vasculature (CD31/MECA-32) and hypoxia (carbonic anhydrase IX8) to identify hypoxic regions. These stains, together with Tc identification, enable identification of Myo[S] treated Tc migration to, and persistence in, areas of low oxygen.

Parts of tumours, spleens, tumour non-draining and tumour draining lymph nodes are harvested and processed for FACS analysis. Tc purified from the tumour are analysed for their viability, ability to kill tumour cells in vitro and expression of CD44, CD62L, PD1, Annexin V, IFNγ and TNFα. Additionally, the phenotype of the tumour cells is analysed for MHC Class I and Class II as well as PDL1.

### Examples—Results and Discussion

Percentage of Cells Labelled with Construct

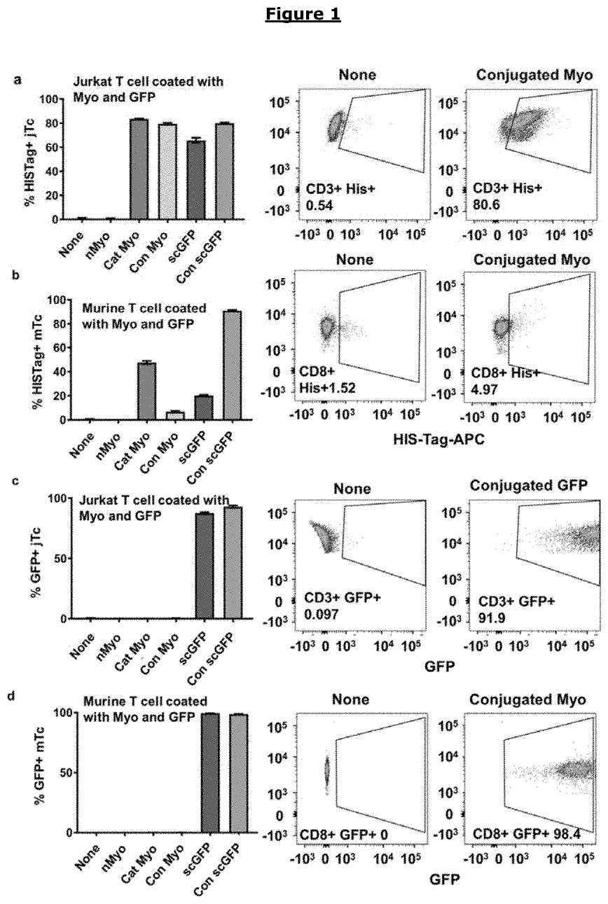

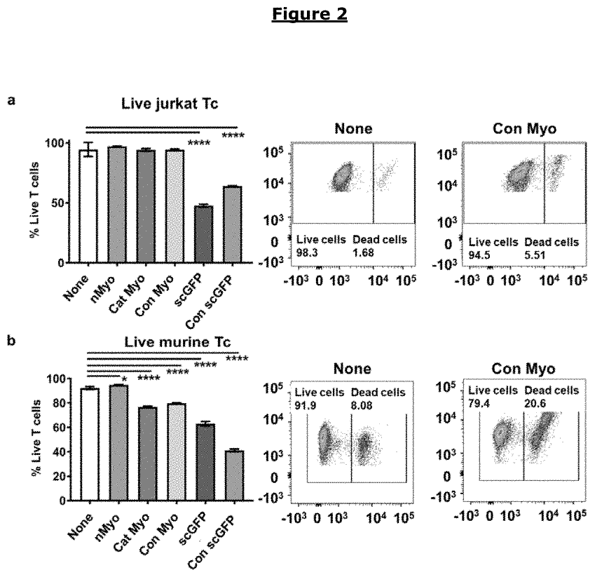

Coated live cells were immediately stained with fluorescently conjugated antibodies and viability dyes as described above and then analysed, by flow cytometry, for viability and presence of construct. Both GFP and HIS-TAG were detectable on the surface of Tc after 3 washes suggesting that they had bound tightly to the cell surface (FIG. 1a-d). The percentage of jTc that had bound cationised myoglobin (83.5%+/−0.4) and conjugated myoglobin (79.4%+/−0.8) were equivalent (FIG. 1a) however there was a significant decrease in the percentage of mTc that bound to conjugated myoglobin (6.7%+/−0.8), compared to cationised myoglobin (47.7%+/−1.4)(FIG. 1c). Without wishing to be bound by theory, the reasons for the differential binding of cationised and conjugated myoglobin to jTc and mTc may be due to variable glycosylation on the cell membrane or differences in the ability to detect HIS-Tag labelled proteins. In support of the latter hypothesis GFP labelling of both jTc and mTc is greater than 87% with both supercharged and conjugated GFP (FIGS. 1b and d) however the equivalent HIS-Tag labelling varies from 20% to 91% suggesting that HIS-Tag labelling is not detecting all GFP labelling and needs further consideration. Without wishing to be bound by theory, it is possible that the low HIS-Tag readout and high GFP signal may be caused by internalisation of the construct, or by binding to the mTc cell in an orientation that masks the HIS-Tag, both of which would not affect the GFP signal but would prevent anti-HIS-Tag antibody binding.

Cell Viability

The viability of jTc incubated without myoglobin or with native, cationised or conjugated myoglobin was above 94%, however viability of jTc with scGFP and conjugated GFP was 47.75% and 63.95% respectively (FIG. 2a) suggesting that GFP but not myoglobin is toxic to jTc at the concentrations tested. The viability of mTc without myoglobin (92.3%+/−1.14) and with native myoglobin (94.65%+/−0.31) was significantly higher than mTc coated with cationised myoglobin (76.78%+/−0.50) and conjugated myoglobin (79.7%+/−0.4) suggesting that cationised and conjugated myoglobin were causing some cell death at the concentrations tested. There was even more cell death in mTc coated with scGFP (63.0%+/−1.9) and conjugated GFP (41.1%+/−1.12) (FIG. 2b).

Together, these results demonstrate good cell surface binding of cationised myoglobin to both jTc and mTc, with minimal cytotoxicity to jTc in particular. Further experiments with mTc are required to optimise the binding of conjugated myoglobin whilst improving cell viability. It is likely that the differential viability of mTc and jTc in response to identical concentrations of cationised and conjugated myoglobin is due to the inherent robustness of the cells, with the jTc cell line being more robust than primary naïve murine Tc.

To ascertain the optimal concentration of surfactant-conjugated supercharged myoglobin (Myo14[S]) for T cell treatment the viability of jTc and primary mTc were tested after coating with varying concentrations of Myo14[S]. The viability of jTc was above 90% at all concentrations tested up to a maximum of 40 μM (FIG. 5b). The viability of primary murine T cells, 1 day after coating with Myo14[S] was 90% or above at all concentrations tested up to 10 μM (FIG. 5c).

Activity—to Cognate Peptide

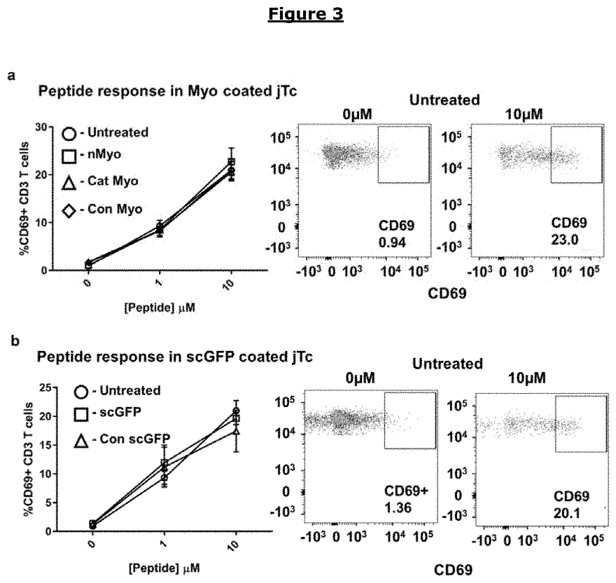

In order to test whether coating jTc with constructs inhibits the engagement of T cell receptors (TCR) with their cognate peptide presented by the major histocompatibility complex (MHC), TCR transgenic Jurkat T cells were utilised, which upregulate CD69 when they recognise cognate peptide presented by C1R-HLA-A2 (C1R) B cells. jTc were coated with myoglobin or GFP constructs and incubated, for 6 hours, with C1R cells that had been previously pulsed with 0 μM, 1 μM or 10 μM of the cognate peptide. The upregulation of CD69, an activation marker, was analysed by flow cytometry. The addition of 1 μM or 10 μM of peptide increased the percentage of cells expressing CD69 from 0.92% (+/−0.06) for untreated jTc to 9.32% (+/−1.16) for 1 μM of peptide and 20.97% (+/−1.80) for 10 μM of peptide (FIGS. 3a and b). There was no significant difference in the increase of CD69 expression after coating with cationised or conjugated myoglobin (FIG. 3a) or supercharged or conjugated GFP (FIG. 3b). The results show that binding of TCR transgenic jTc with the constructs does not inhibit T cell activation which suggests that there is no steric interference caused by the constructs between the TCR-MHC activation complex.

Proliferation and Activity—Under Hypoxic Conditions

mTc were activated under normoxic (22% oxygen) and hypoxic (5% oxygen) conditions after coating with myoglobin constructs. mTc were incubated for 4 days in the presence of anti-CD3 and anti-CD28 antibodies. Together, anti-CD3 and anti-CD28 bind to the TCR and the co-stimulatory molecule, CD28, and cause polyclonal activation of Tc. On day 4, mTc were harvested and analysed by flow cytometry. mTc were gated on CD4 or CD8, to analyse these two subtypes of T cells. Each subtype was analysed for the dilution of cell trace violet, (CTV) a marker of proliferation, and high expression of CD44, a marker of activation.

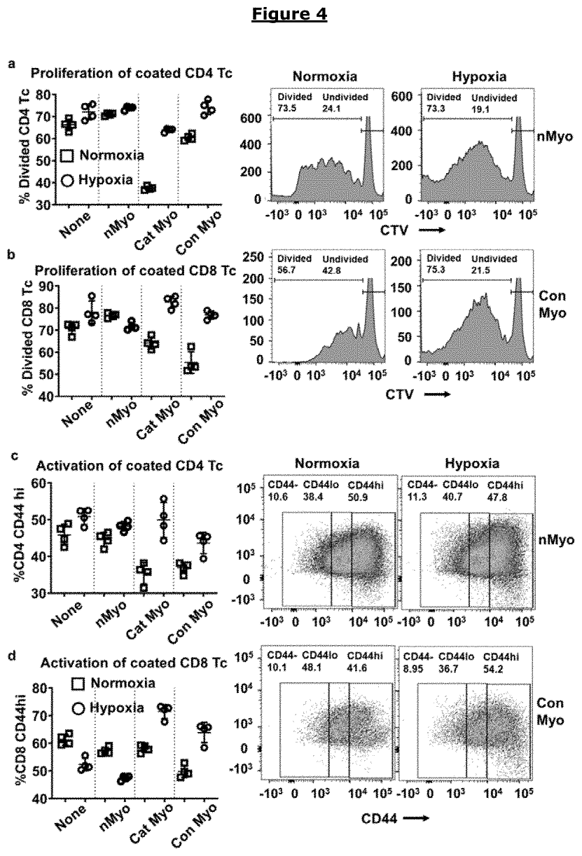

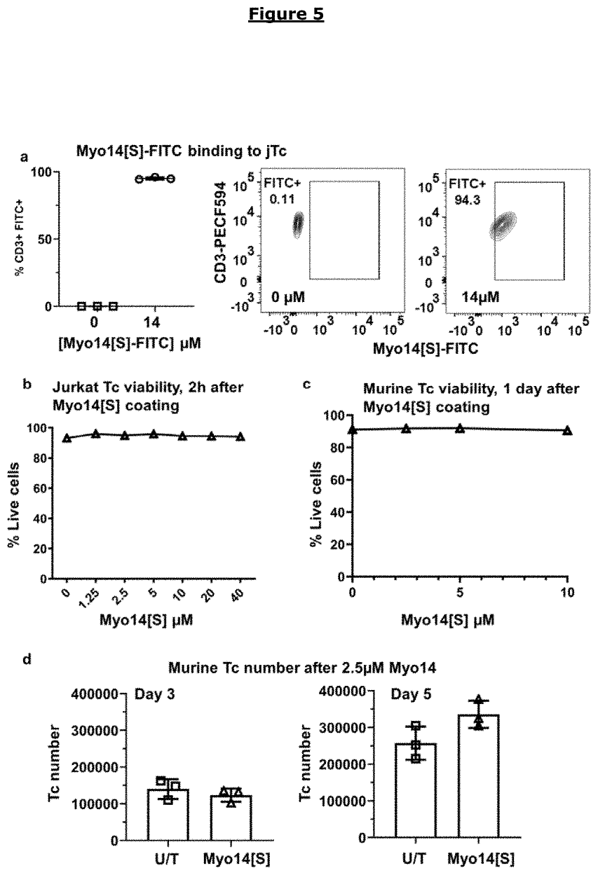

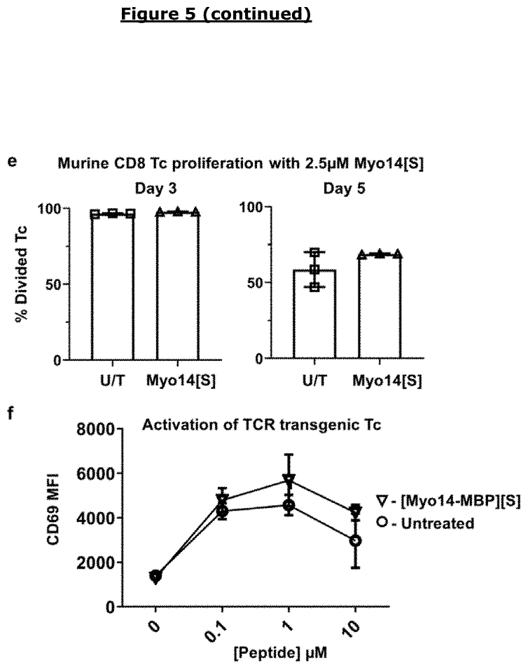

In untreated cells, hypoxia increased the percentage of divided CD4 mTc from 66.23% (+/−2.54) to 72.1% (+/−3.54) and CD8 mTc from 70.75% (+/−2.63) to 78.2% (+/−5.15) suggesting that the hypoxic environment increases Tc proliferation (FIGS. 4a and b). Under normoxic conditions, CD4 and CD8 mTc coated with cationised or conjugated myoglobin all had a significantly lower percentage of divided cells than their untreated or native myoglobin treated counterparts. This observation can be rationally explained by the decreased viability of these cells after coating with the myoglobin constructs (FIG. 2b) before activation. However, when coated CD4 and CD8 Tc were cultured under hypoxic conditions there was a higher percentage of divided mTc when compared to normoxic conditions suggesting that the presence of myoglobin and/or hypoxia was rescuing the proliferative defects in these cells.

In untreated CD4 Tc, when compared to normoxia, hypoxia increased the percentage of activated CD4 CD44hi Tc from 45.9% (+/−2.86) to 50.83% (+/−2.16). CD4 Tc coated with cationised or conjugated myoglobin and activated under normoxic conditions had a significantly lower percentage of CD4 CD44hi Tc than untreated or native myoglobin treated Tc, however the same cells activated under hypoxic conditions had an equivalent percentage of CD4 CD44hi Tc (FIG. 4c) when compared to untreated and native myoglobin treated mTc.

In untreated CD8 Tc, when compared to normoxia, hypoxia significantly (p=0.001) decreased the percentage of CD8 CD44hi Tc from 61.25% (+/−1.93) to 52.33% (+/−2.27). The addition of conjugated (49.83%+/−2.21), but not cationised (58.08%+/−1.34), myoglobin resulted in a reduction in the percentage of CD8 CD44hi Tc under normoxic conditions, when compared to untreated controls (61.25%+/−1.93). However, the addition of cationised or conjugated myoglobin, under hypoxic conditions, reversed the hypoxia induced reduction in CD8 Tc activation. In fact, the addition of both forms of myoglobin significantly increased the percentage of CD8 CD44hi Tc from 52.33% (+/−2.27) for untreated CD8 Tc to 71.5% (+/−2.50) for cationised myoglobin coated CD8 Tc and 63.88% (+/−3.67) for conjugated myoglobin coated CD8 Tc (FIG. 4d). It is unclear why myoglobin constructs cause a decrease in CD44hi Tc under normoxic, but not hypoxic conditions. It is possible that the original decrease in the viability of Tc may be involved. It is also possible that the constructs may be interfering with T cell signalling, to some extent, through the TCR or CD28 in a way that is only important under normoxic conditions. However, the demonstration that treatment with myoglobin rescues the activated phenotype of CD8 Tc under normoxic conditions is greatly encouraging as CD8 Tc are an important target of further development in both CAR-T cell and TIL therapies.

Proliferation

The proliferation and viability of in vitro activated mTc, 3 and 5 days after staining with Cell Trace Violet and coating with 2.5 μM Myo14[S] was evaluated. There was no significant difference in the number of live, activated T cells at day 3 or day 5 after coating with 2.5 μM Myo14[S] when compared to untreated (U/T) controls (FIG. 5d). Moreover, in the same experiment, the percentage of mTc that had undergone one or more divisions was not significantly different in Myo14[S] treated and untreated controls (FIG. 5e).

Antigen-Specific Activation

The ability of T cell receptor (TCR) transgenic Jurkat T cell to become activated upon recognition of their cognate MHC-peptide complex was tested in a CD69 activation assay. [Myo14-MBP][S] was used in this assay because the Maltose binding protein (MBP) was used as a surrogate for any other protein that can be linked to Myo14, including, but not restricted to, Cytokines such as IL-2, IL-15 and IL-17, T cell receptors such as PD-1, CTLA-4, TIM-3 or enzymes such as metalloproteinases. The upregulation of CD69 on jTc coated with [Myo14-MBP][S], in response to increasing peptide concentrations was not significantly different from untreated controls. These results demonstrate that coating with Myo14[S] linked to another protein (in this case MBP) does not interfere with normal jTc to target cell protein interactions and thus should not interfere with normal T cell signalling (FIG. 5f).

Exhaustion-Marker Profiling

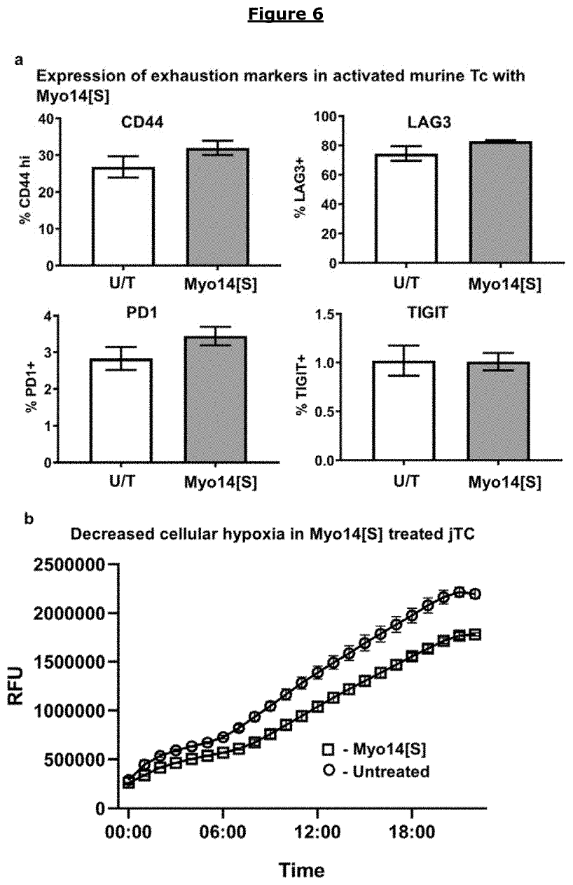

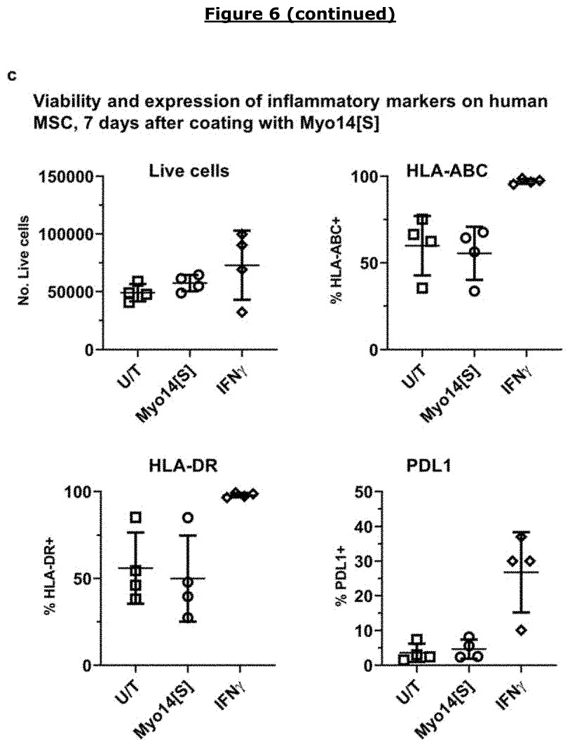

In order to demonstrate that naïve mTc did not become stressed in the presence of surfactant-conjugated supercharged myoglobin (Myo14[S]), purified mTc were coated with 2.5 μM Myo14[S] and stimulated with αCD3/CD28 and IL-2 for 3 days. On day 3, activated mTc were harvested and analysed for their expression of the activation marker CD44 and exhaustion/activation markers LAG3, PD1 and TIGIT. There was no significant difference between untreated and Myo14[S] treated cells for any of the markers tested (FIG. 6a).

Hypoxia Dye Assay

Surfactant-conjugated supercharged myoglobin (Myo14[S]) is an oxygen carrying molecule and so the inventors sought to determine whether oxygen carried by Myo14[S] could be supplied to Tc. jTc were stained with Image-iT Green hypoxia reagent and then were left untreated, or were treated with 10 μM Myo14[S]. Stained and coated jTc were transferred to a Cytation plate reader and fluorescence was monitored for 22 hr at 5% Carbon dioxide and 0.5% Oxygen. The relative fluorescence and therefore the internal hypoxic state of the untreated jTc increases at a greater rate and reaches a higher level than the Myo14[S] treated jTc suggesting that Myo14[S] is supplying oxygen to the jTc (FIG. 6b).

hMSC Inflammation Assay

The technology described herein can be applied to numerous cell types and disease indications. The inventors have demonstrated that surfactant-conjugated supercharged myoglobin (Myo14[S]) can be coated onto human mesenchymal stem cells (hMSC). hMSC were coated with 5 μM Myo14[S] and then grown in vitro for 7 days. On day 7 hMSC were harvested, live cells were counted and then cells were stained with antibodies for lineage markers CD73 and CD105 and inflammatory markers HLA-ABC, HLA-DR and PD-L1. As a control for the upregulation of inflammatory markers cells were also cultured with the inflammatory cytokine IFNγ. There was no significant difference between the cell number or percentage of cells expressing HLA-ABC, HLA-DR and PD-L1 in untreated and Myo14[S] treated hMSC (FIG. 6c).

Table 4 provides the amino acid sequences of a series of charge-modified (supercharged) myoglobins generated using the above-described techniques:

TABLE 4
Amino acid sequences of charge-modified myoglobins and charge-modified GFP

| SEQ | | |
|---|---|---|
| ID NO | Description | Sequence |
| 1 | 1st mutant- | MHHHHHHGSSGENLYFQGLSDGEWQLVLNVWGKVEADIPGHGQEVL |
| | mutations | IRLFKGHPKTLKKFDRFKHLKSKKKMKASEKLKKHGATVLTALGGILKK |
| | informed by | KGHHEAKIKPLAQSHATKHKIPVKYLKFISKAIIKVLQSKHPGDFGKKA |
| | chemical | QGAMNKALKLFRKKMASNYKEL |
| | cationisation | |
| 2 | Max-all acid | MHHHHHHGSSGENLYFQGLSDGEWQLVLNVWGKVEAKIPGHGQEVL |
| | residues | IRLFKGHPKTLKKFDRFKHLKSKKKMKASKKLKKHGATVLTALGGILKK |
| | modified to | KGHHKAKIKPLAQSHATKHKIPVKYLKFISKAIIQVLQSKHPGKFGAKA |
| | lysines | QGAMNKALKLFRKKMASNYKKL |
| 3 | Polarised- | MHHHHHHGSSGENLYFQGLSKGKWQLVLNVWGKVKAKIPGHGQKV |
| | mutations | LIRLFKGHPKTLKEFKRFKHLKSKKKMKASKKLKKHGATVLTALGGILK |
| | made to acids | KKGHHEAKIEPLAQSHATEHEIPVEYLEFISKAIIQVLQSKHPGKFGAKA |
| | excluding 10 Å | QGAMNKALKLFRKDMASNYEEL |
| | radius around | |
| | C-terminal | |
| 4 | Rosetta | MHHHHHHGGGGSENLYFQGLSDGEWQLVLNVWGKVEADIPGHGQE |
| | | VLIRLFKGHPETLKKFDRFKKLKSEDKMKKSEDLKKHGATVLKRLGGIL |
| | | KKKGRHEAKIKPLAQRHAKKHKIPVKYLEFRSEAIIRVLRSKHPGDFGA |
| | | DAQGAMNKALELFRKDMASNYKELGFQG |
| 5 | AVNAPSA | MHHHHHHGGGGSENLYFQGLSKGEWKLVLNVWGKVEADIPGHGQE |
| | | VLIRLFKGHPKTLKKFKRFKHLKSEKKMKASKDLKKHGATVLTALGGIL |
| | | KKKGHHKAEIKPLAQSHATKHKIPVKYLEFISEAIIQVLQSKHPGKFGA |
| | | KAQGAMNKALELFRKDMASNYKKLGFQG |
| 6 | TaB | MHHHHHHGGGGSENLYFQGLSDGEWRLVLKVWGKVERDIPGHGQE |
| | | VLIRLFKGHPETLKKFDRFKHLKSEREMKASKDLKKHGATVLTALGGIL |
| | | KKKGHHEAEIKPLAKSHATKHKIPVKYLKFISKAIIQVLQSKHPGDFGA |
| | | RAQGAMNKALELFRKDMARNYKKLGFQG |
| 7 | Consensus B- | MHHHHHHGGGGSENLYGLSDGEWRLVLKVWGKVERDIPGHGQEVLI |
| | The same as | RLFKGHPETLKKFDRFKHLKSRDEMKASEKLKKHGATVLTALGGILKK |
| | TaB, but | KGHHEAEIKPLAKSHATKHKIPVKYLKFISKAIIQVLQSKHPGDFGADA |
| | adjusted to | QGAMNKALELFRKDMASKYKKLGFQG |
| | exclude | |
| | conserved | |
| | domains | |
| 8 | Myo7 | MHHHHHHGSGGLSDGEWQLVLKVWGKVEADIPGHGQEVLIRLFKGH |
| | | PETLEKFDRFKHLKSEDEMKASEDLKKHGATVLTALGKILKKKGHHEA |
| | | EIKPLAQSHATKHKIPVKYLKFISEAIIKVLQSKHPGDFGADAQGAMKK |
| | | ALELFRKDMASKYKELGFQG |
| 9 | Myo14 | MHHHHHHGSGGLSDGEWQLVLKVWGKVEADIPGHGQEVLIRLFKGH |
| | | PETLKKFDRFKHLKSEDEMKASEDLKKHGATVLKKLGKILKKKGKHEA |
| | | EIKPLAQSHATKHKIPVKYLKFISEAIIKVLQSKHPGDFGADAQGAMKK |
| | | ALKLFRKDMASKYKELGFQG |
| 10 | cMyo9 | MHHHHHHGSGGLSDGEWQLVLKVWGKVEADIPGHGQEVLIRLFKGH |
| | | PETLEKFDRFKHLKSEDEMKRSEDLKKHGATVLKALGKILKKKGHHEA |
| | | EIKPLAQSHATKHKIPVKYLKFISEAIIKVLQSKHPGDFGADAQGAMKK |
| | | ALELFRKDMASKYKELGFQG |
| 11 | cMyo14 | MHHHHHHGSGGLSDGEWQLVLKVWGKVEADIPGHGQEVLIRLFKGH |
| | | PETLEKFDRFKKLKSEDEMKRSEDLKKHGATVLKKLGKILKKKGKHEA |
| | | EIKPLAQSHATKHKIPVKYLKFISEAIIKVLQSKHPGDFGADAQGAMKK |
| | | ALKLFRKDMASKYKELGFQG |
| 12 | cMyo15 | MHHHHHHGSGGLRDGEWQLVLKVWGKVEADIPGHGQEVLIRLFKGH |
| | | PETLEKFDRFKKLKSEDEMKRSEDLKKHGATVLKKLGKILKKKGKHEA |
| | | EIKPLAQSHATKHKIPVKYLKFISEAIIKVLQSKHPGDFGADAQGAMKK |
| | | ALKLFRKDMASKYKELGFQG |
| 13 | His_MBP_PD1_ | MHHHHHHKIEEGKLVIWINGDKGYNGLAEVGKKFEKDTGIKVTVEHP |
| | Myo14 | DKLEEKFPQVAATGDGPDIIFWAHDRFGGYAQSGLLAEITPDKAFQDK |
| | | LYPFTWDAVRYNGKLIAYPIAVEALSLIYNKDLLPNPPKTWEEIPALDKE |
| | | LKAKGKSALMFNLQEPYFTWPLIAADGGYAFKYENGKYDIKDVGVDNA |
| | | GAKAGLTFLVDLIKNKHMNADTDYSIAEAAFNKGETAMTINGPWAWS |
| | | NIDTSKVNYGVTVLPTFKGQPSKPFVGVLSAGINAASPNKELAKEFLEN |
| | | YLLTDEGLEAVNKDKPLGAVALKSYEEELVKDPRIAATMENAQKGEIMP |
| | | NIPQMSAFWYAVRTAVINAASGRQTVDEALKDAQTNSSSNNNNNNN |
| | | NNNLGENLYFQGSGWNPPTFSPALLVVTEGDNATFTCSFSNTSESFVL |
| | | NWYRMSPSNQTVKLAAFPEDRSQPGQDSRFRVTQLPNGRDFHMSVV |
| | | RARRNDSGTYLCSAISLAPKAQIKESLRAELRVTERRAEVPTAHPSPSP |
| | | GGGGSGGGGSGGGGSGLSDGEWQLVLKVWGKVEADIPGHGQEVLI |
| | | RLFKGHPETLKKFDRFKHLKSEDEMKASEDLKKHGATVLKKLGKILKK |
| | | KGKHEAEIKPLAQSHATKHKIPVKYLKFISEAIIKVLQSKHPGDFGADA |
| | | QGAMKKALKLFRKDMASKYKELGFQG |
| 14 | MBP_PD1_ | MKIEEGKLVIWINGDKGYNGLAEVGKKFEKDTGIKVTVEHPDKLEEKF |
| | Myo14 | PQVAATGDGPDIIFWAHDRFGGYAQSGLLAEITPDKAFQDKLYPFTWD |
| | | AVRYNGKLIAYPIAVEALSLIYNKDLLPNPPKTWEEIPALDKELKAKGKS |
| | | ALMFNLQEPYFTWPLIAADGGYAFKYENGKYDIKDVGVDNAGAKAGLT |
| | | FLVDLIKNKHMNADTDYSIAEAAFNKGETAMTINGPWAWSNIDTSKV |
| | | NYGVTVLPTFKGQPSKPFVGVLSAGINAASPNKELAKEFLENYLLTDEG |
| | | LEAVNKDKPLGAVALKSYEEELVKDPRIAATMENAQKGEIMPNIPQMS |
| | | AFWYAVRTAVINAASGRQTVDEALKDAQTNSSSNNNNNNNNNNLGE |
| | | NLYFQGSGWNPPTFSPALLVVTEGDNATFTCSFSNTSESFVLNWYRMS |
| | | PSNQTVKLAAFPEDRSQPGQDSRFRVTQLPNGRDFHMSVVRARRNDS |
| | | GTYLCSAISLAPKAQIKESLRAELRVTERRAEVPTAHPSPSPGGGGSGG |
| | | GGSGGGGSGLSDGEWQLVLKVWGKVEADIPGHGQEVLIRLFKGHPE |
| | | TLKKFDRFKHLKSEDEMKASEDLKKHGATVLKKLGKILKKKGKHEAEI |
| | | KPLAQSHATKHKIPVKYLKFISEAIIKVLQSKHPGDFGADAQGAMKKAL |
| | | KLFRKDMASKYKELGFQG |
| 15 | scGFP | MASKGERLFRGKVPILVELKGDVNGHKFSVRGKGKGDATRGKLTLKFI |
| | | CTTGKLPVPWPTLVTTLTYGVQCFSRYPKHMKRHDFFKSAMPKGYVQE |
| | | RTISFKKDGKYKTRAEVKFEGRTLVNRIKLKGRDFKEKGNILGHKLRYN |
| | | FNSHKVYITADKRKNGIKAKFKIRHNVKDGSVQLADHYQQNTPIGRGP |
| | | VLLPRNHYLSTRSKLSKDPKEKRDHMVLLEFVTAAGIKHGRDERYK |

1. An antitumour cell, liposome or micelle, comprising at least one charge-modified globin associated with the membrane of the cell, liposome or micelle.

2. A cell according to claim 1, wherein the cell is an immune cell, preferably a tumour-infiltrating immune cell, more preferably a lymphocyte, neutrophil, dendritic cell or macrophage.

3. A cell according to claim 1 or 2, wherein the cell is a cytotoxic T cell, natural killer T cell or natural killer cell.

4. A cell according to any preceding claim, wherein the cell is a T cell.

5. A liposome or micelle according to claim 1, wherein the liposome or micelle comprises a therapeutic agent, preferably wherein the therapeutic agent is a checkpoint inhibitor, immunotherapeutic or chemotherapeutic agent.

6. A cell, liposome or micelle according to any preceding claim, wherein the globin is haemoglobin, myoglobin, neuroglobin, or cytoglobin, preferably myoglobin.

7. A cell, liposome or micelle according to any preceding claim, wherein the globin is linked to a secondary antitumour molecule, or a reactive functional group for linking to a secondary antitumour molecule, preferably wherein the secondary antitumour molecule is any one of an antibody, lectin, integrin or adhesion molecule; and/or preferably wherein the secondary antitumour molecule is any one of: (1) a tumour cell binding molecule; (2) a checkpoint inhibitor; (3) an enzyme that remodels the extracellular matrix of a tumour; or (4) an enzyme that metabolises tumour-associated compounds.

8. A cell, liposome or micelle according to claim 7, comprising a fusion protein comprising the globin and the secondary anti-cancer molecule.

9. A cell, liposome or micelle according to any preceding claim, wherein the globin is a cationised or anionised globin.

10. A cell, liposome or micelle according to any preceding claim, wherein the globin comprises a polymer surfactant coating.

11. A pharmaceutical composition comprising the antitumour cell, liposome or micelle according to any preceding claim, further comprising a pharmaceutically acceptable carrier, diluent or vehicle.

12. A cell, liposome or micelle according to any of claims 1-10, or the pharmaceutical composition according to claim 11, for use in the treatment of cancer.

13. A method of making the antitumour cell, liposome or micelle according to any of claims 1-10, comprising
a) providing a charge-modified globin; and
b) contacting the antitumour cell, liposome or micelle with the globin.

14. The method of claim 13, wherein step (a) comprises providing a charge-modified globin and a polymer surfactant under conditions which enable electrostatic conjugation of the polymer surfactant with the globin.

15. The method of claim 13 or 14, wherein a globin is converted to the charge-modified globin by a method comprising:
i) mixing a solution of globin with a pH-neutralised solution of N,N′-dimethyl-1,3-propanediamine (DMPA) or analogue thereof and optionally adjusting the mixture to pH 5-7;
ii) subsequently or concurrently adding a carbodiimide such as N-(3-dimethylaminopropyl)-N′ethylcarbodiimide hydrochloride (EDC) and adjusting the mixture to pH 4-7;
iii) agitating the mixture from (ii) for 1-30 hours at pH 4-7, at a temperature of 0-25° C.;
iv) dialysing the protein in the mixture from (iii) against water or buffer for at least 4 hours at pH 6.5-8.5;
v) if necessary, adjusting the pH of the mixture from (iv) to pH 6.5-8.5.

16. The method of claim 13 or 14, wherein the charge-modified globin is obtained by a method comprising expression of a recombinant DNA sequence encoding for the charge-modified globin.

17. A method of treating cancer, comprising administration of the cell, liposome or micelle according to any one of claims 1 to 10, or the pharmaceutical composition according to claim 11, to a patient in need thereof.

18. The use according to claim 12, or the method according to claim 17, wherein the cancer is a solid tumour cancer.

19. The use according to claim 12 or 18, or the method according to claim 17 or 18, wherein the cancer is selected from: breast, colorectal, prostate, lung, stomach, liver, oesophageal, cervical, or pancreatic cancer.

20. A polypeptide comprising the charge modified globin sequence of any of SEQ ID NOs: 1-14 or a functional variant of any of these having at least about 60% sequence identity with the non-variant globin sequence.
